(19) United States
(12) Patent Application Publication
(10) Pub. No.: US 2024/0032843 A1
(43) Pub. Date: Feb. 1, 2024

(54) INTRAOPERATIVE ALARM MONITOR

(71) Applicant: Libra Science Ltd., Road Town, Tortola (VG)

(72) Inventor: Shlomo BEN-HAIM, Milan (IT)

(73) Assignee: Libra Science Ltd., Road Town, Tortola (VG)

(21) Appl. No.: 18/265,465

(22) PCT Filed: Dec. 6, 2021
(86) PCT No.: PCT/IB2021/061368

§ 371 Date: Jun. 6, 2023

Related U.S. Application Data
(60) Provisional application No. 63/171,634, filed on Apr. 7, 2021.
(60) Provisional application No. 63/121,940, filed on Dec. 6, 2020.

(51) Int. Cl. A61B 5/349; A61B 5/00; A61B 18/14

(52) U.S. Cl. CPC A61B 5/349; A61B 5/726; A61B 18/1492; A61B 5/746

(57) ABSTRACT

Automatic intraoperative monitoring of new onset conduction disturbances (NOCD) comprising comparison of metric values derived from an ECG signal recording to reference values. An alarm is raised when the comparison result indicates a new onset conduction disorder. Other indications of the comparison may also be presented. Reference values may derive, for example, from a baseline ECG signal recording, and/or from a database of previously observed associations between ECG signals and NOCD. In some embodiments, comparison is also made to threshold values defined by clinical heart block criteria. Baseline and clinical threshold values may define a range used in the comparison. Comparison may combine ranges; for example, using Boolean logic. Comparison and/or alarm triggering is optionally modulated by a database of previously observed associations between non-ECG data and NOCD. Optionally, a user is enabled to select alarm triggering conditions through a user interface.

### RELATED APPLICATIONS

This application claims the benefit of priority under 35 USC § 119(e) of U.S. Provisional Patent Application No. 63/121,940 filed Dec. 6, 2020; as well as U.S. Provisional Patent Application No. 63/171,634 filed Apr. 7, 2021; the contents of each of which are incorporated herein by reference in their entirety.

### FIELD AND BACKGROUND OF THE INVENTION

The present invention, in some embodiments thereof, relates to the field of transcatheter surgeries and more particularly, but not exclusively, to structural heart disease interventions.

Treatments have been developed and continue to be developed for a wide spectrum of structural heart diseases (SHD). These treatments commonly involve implantations and/or physical manipulations of heart tissue, often via transcatheter delivery.

There is an incidence of new onset conductance disturbances (NOCDs) associated with certain of these treatments; for example, in procedures and/or implantations to correct: atrial and ventricular septal defects (e.g., via percutaneous device-based closures), aortic stenosis (e.g., via transcatheter aortic valve replacement, TAVR), and tricuspid regurgitation (e.g., via transcatheter implantation of devices for annuloplasty, coaptation, and/or valve replacement).

In particular, the location of components of the heart's conduction system (e.g., atrioventricular node (AVN), bundle of His, and bundle branches) with respect to the inferior aspect of the interatrial septum, membranous interventricular septum, and coronary sinus have important implications for the risk of NOCDs following various percutaneous structural heart interventions.

### SUMMARY OF THE INVENTION

According to an aspect of some embodiments of the present disclosure, there is provided a method of presenting an alarm indicative of a risk of new onset conduction disturbance induced by a transcatheter heart procedure, the method including: obtaining, by a computer processor, a reference value of a metric indicative of a heart conduction block; comparing, during a monitoring period including at least a portion of the transcatheter heart procedure, a test value of the metric to the reference value of the metric; wherein the test value is obtained by analyzing ECG signal recordings recorded from a patient who undergoes the transcatheter heart procedure; determining a result of the comparing to be indicative of a new onset of heart conduction block; and presenting the alarm, based on the determining.

According to some embodiments of the present disclosure, the comparing is carried out on a plurality of test values during the monitoring period before being carried out on the test value yielding the determining.

According to some embodiments of the present disclosure, obtaining the reference value includes selecting from predetermined values, each associated with non-ECG factors characterizing a transcatheter heart procedure.

According to some embodiments of the present disclosure, obtaining the reference value includes analyzing an ECG signal recording recorded from the patient during the performance of the transcatheter heart procedure and before the recording the ECG signal to obtain the test value.

According to some embodiments of the present disclosure, obtaining the reference value includes analyzing an ECG signal recording recorded from the patient before the performance of the transcatheter heart procedure.

According to some embodiments of the present disclosure, the metric is obtained using an interval defined between two landmark ECG features.

According to some embodiments of the present disclosure, the metric is derived from an ECG-based axis lead determination.

According to some embodiments of the present disclosure, the comparing includes comparing the test values of the metric to a threshold value indicative of clinically significant heart conduction block.

According to some embodiments of the present disclosure, the metric is derived from an ECG-based vector electrogram.

According to some embodiments of the present disclosure, the comparing includes one or more of: determining a deviation of a position of a loop of the vector electrogram, used as the metric, away from an alert range of positions at least partially defining the reference metric; determining a deviation of a derivative of the loop of the vector electrogram, used as the metric, away from an alert range of derivative values at least partially defining the reference metric; and determining a deviation of timing within the loop of the vector electrogram, used as the metric, away from an alert range of time values at least partially defining the reference metric.

According to some embodiments of the present disclosure, the comparing is performed for test values and reference values of a plurality of different metrics, and the metrics are defined to use one or more of: one or more ECG leads; one or more determinations of ECG lead axis; and an ECG-based vector electrogram.

According to some embodiments of the present disclosure, the comparing uses the threshold setting to distinguish a difference or similarity between the test value and the reference value; and the presenting the alarm is based on the distinguished difference or similarity.

According to some embodiments of the present disclosure, the threshold setting is derived from a group of predetermined criteria for establishing the presence of a clinical condition.

According to some embodiments of the present disclosure, the method includes accessing a user-defined setting, and using the user-defined setting to control sensitivity of the alarm to the comparing.

According to some embodiments of the present disclosure, the method includes displaying a graph showing two sets of values, which respectively indicate the reference value and the test value; and which are sets of at least one of lead, lead axis, and vector electrogram data values.

According to some embodiments of the present disclosure, the comparing uses the values of non-ECG factors associated to the transcatheter heart procedure to select the reference value.

According to some embodiments of the present disclosure, the method includes: accessing, by the computer processor, a database indicative of associations of each of a plurality of transcatheter heart procedures with the occurrence of conduction block as a result of the procedure, and values of the non-ECG factors characterizing the procedure; and using the database to obtain the reference value of the metric.

According to some embodiments of the present disclosure, the method includes: accessing, by the computer processor, a baseline portion of the ECG signal recording; and generating the reference value of the metric from the baseline portion.

According to some embodiments of the present disclosure, the generating the reference metric includes comparing a value of the metric calculated from the template ECG signal to a value of the metric calculated from the ongoing portion of the ECG signal.

According to some embodiments of the present disclosure, the changed result of the comparing includes a new difference between the test value and the reference value, and the alarm is presented based on the detected new difference.

According to some embodiments of the present disclosure, the method includes accessing, by the computer processor, a database indicative of a range of values of the metric associated with sub-threshold heart conduction block; wherein the changed result of the comparing comprises a new correspondence of the test value to the range of values.

According to some embodiments of the present disclosure, the transcatheter heart procedure includes a structural heart disease intervention.

According to some embodiments of the present disclosure, the structural heart disease intervention includes any one of the group consisting of: transcatheter aortic valve replacement, septal defect repair device placement, and annuloplasty.

According to some embodiments of the present disclosure, the method includes the processor accessing an indication of the time of a potentially risky phase of the transcatheter procedure, wherein the comparing and/or the presenting is based in part on the indication.

According to an aspect of some embodiments of the present disclosure, there is provided a device for intraoperative monitoring of new onset conduction disturbances, the device including: a processor and digital memory including processor instructions which instruct the processor to: access a portion of an ongoing ECG signal recording recorded from a patient during the performance of a transcatheter heart procedure; compare a test value of a metric to a reference value of the metric, the test value being calculated from the portion of the ongoing ECG recording; and an indicator device, coupled to the processor, and configured to present an alarm indicative of a result of the comparison; wherein the metric is indicative of a level of sub-threshold heart conduction block.

According to some embodiments of the present disclosure, together with ECG circuitry is configured to record the ECG.

According to an aspect of some embodiments of the present disclosure, there is provided a method of medical monitoring including: detecting automatically, during recording of an ECG from a patient, an onset of NOCD indicated by the ECG signal recording; and presenting, immediately upon detection of the onset of the NOCD, an automatically generated NOCD onset alert to a human medical caretaker via an electronic interfacing device.

According to some embodiments of the present disclosure, the NOCD onset alert includes an indication of the time of NOCD onset.

According to some embodiments of the present disclosure, the NOCD onset alert includes an indication estimating the severity of the NOCD.

According to some embodiments of the present disclosure, the NOCD onset alert is presented to a human medical caretaker outside the presence of the patient.

According to some embodiments of the present disclosure, the NOCD onset alert is presented to a human medical caretaker performing a transcatheter heart treatment of the patient.

According to some embodiments of the present disclosure, the detecting is performed during a period following transcatheter heart treatment of the patient.

According to some embodiments of the present disclosure, the detecting uses a value determined by at least one of a prior condition of the patient and the parameters of the treatment.

According to an aspect of some embodiments of the present disclosure, there is provided a method of medical monitoring, including: inducing a potential new onset conduction delay in the heart conduction system of a patient during a medical treatment; detecting the new onset conduction delay, based on analysis of ECG signal recordings made of the heart during the medical treatment; and presenting an alarm, warning of the new onset conduction delay.

According to an aspect of some embodiments of the present disclosure, there is provided a method of monitoring the heart conduction system of a patient, including: accessing data indicative of a baseline state of the patient's heart conduction system; generating, using the data indicative of a baseline state, range parameters indicative of sub-threshold heart conduction block; accessing ongoing ECG data monitored from the patient; assigning values to the range parameters based on the ongoing ECG data and the data indicative of the baseline state; calculating an indication of sub-threshold heart conduction block using the values assigned to the range parameters; and presenting the indication.

According to some embodiments of the present disclosure, the ongoing ECG data is accessed during a medical procedure including an action that might induce conduction block.

According to some embodiments of the present disclosure, the data indicative of a baseline state of the patient's heart conduction system comprise baseline ECG data selected from the ongoing ECG data measured during the medical procedure.

According to some embodiments of the present disclosure, the baseline ECG data are selected from a portion of the ongoing ECG data measured before the action that might induce the heart conduction block is taken.

According to some embodiments of the present disclosure, the data indicative of a baseline state of the patient's heart conduction system comprise baseline ECG data measured before the medical procedure.

According to some embodiments of the present disclosure, the generating range parameters includes defining, for each of the range parameters: a range value indicative of relatively lower sub-threshold heart conduction block from the baseline ECG data; and a range value indicative of relatively greater sub-threshold heart conduction block, derived from threshold values of one or more predetermined criteria.

According to some embodiments of the present disclosure, the calculating combines the range parameters according to logic defined for combining the threshold values of the one or more predetermined criteria, and wherein the logic is transformed from Boolean operations combining threshold parameters into other operations combining range parameters.

According to some embodiments of the present disclosure, the other operations comprise one or more of minimum value and maximum value operations.

According to some embodiments of the present disclosure, the one or more predetermined criteria define the threshold values with reference to signal features of leads of a standard 12-lead ECG.

According to some embodiments of the present disclosure, the ongoing ECG data are obtained from the patient using a set of electrodes having less electrodes than defined for performing a standard 12-lead ECG.

According to some embodiments of the present disclosure, the set of electrodes consists of four electrodes.

According to some embodiments of the present disclosure, data indicative of a baseline state of the patient's heart conduction system comprise baseline ECG data measured before the medical procedure wherein the baseline ECG data were collected using a standard 12-lead ECG.

According to some embodiments of the present disclosure, the four electrodes correspond to the arm and leg electrodes of the standard 12-lead ECG, and one of the chest electrodes.

According to some embodiments of the present disclosure, the chest electrode is the V6 electrode.

According to some embodiments of the present disclosure, the calculating includes transforming the ongoing ECG data into leads corresponding to a standard 12-lead ECG.

According to some embodiments of the present disclosure, the transforming includes performing a modified inverse Dower transform to produce a set of leads corresponding to Frank vector cardiography leads, and performing a Dower transform on the produced set of leads to yield the leads corresponding to a standard 12-lead ECG.

According to some embodiments of the present disclosure, calculating the indication includes combining the range parameters into a single range parameter having: a first value indicating a least amount of sub-threshold heart conduction block when each of the range parameters has a value equal to its baseline value; a second value indicating the existence of at least threshold level heart conduction block for one or more other value combinations of the range parameters; and one or more additional values in between the first and second values.

According to some embodiments of the present disclosure, the first value is a minimum value of the single range parameter, and the second value is a maximum value of the single range parameter.

According to some embodiments of the present disclosure, the second value is a minimum value of the single range parameter, and the first value is a maximum value of the single range parameter.

According to some embodiments of the present disclosure, the indication is indicative of heart conduction block attributed to a specific structure of the heart conduction system.

According to some embodiments of the present disclosure, presenting the indication includes marking a visual representation of a heart conduction system with a mark presented at the specific structure of the heart conduction system and having a visual characteristic indicative to the indication.

According to an aspect of some embodiments of the present disclosure, there is provided a method of monitoring heart conduction system of a patient during a transcatheter intervention procedure within the heart of a patient, the method including: receiving ongoing ECG data measured from the patient during the intervention; evaluating the ongoing ECG data to obtain an indication of sub-threshold heart conduction block; and presenting the indication; wherein the ongoing ECG data are evaluated using: criteria for evaluating existence of a heart conduction block, the criteria being predetermined based on ECG data collected from a population of patients, and baseline ECG data measured from the patient before the ongoing ECG data were measured.

According to some embodiments of the present disclosure, the baseline ECG data were measured from the patient at rest.

According to some embodiments of the present disclosure, evaluating the ongoing ECG data includes: calculating the indication of the sub-threshold heart conduction block based on a range parameter which changes monotonically as a feature of the ongoing ECG data approaches a threshold value used to define the criteria.

According to some embodiments of the present disclosure, evaluating the ongoing ECG data includes: transforming a threshold parameter used to define a predetermined criteria into a range parameter; and calculating the indication of the sub-threshold heart conduction block based on the range parameter.

According to some embodiments of the present disclosure, transforming a threshold parameter used to define a predetermined criteria to a range parameter includes: establishing a range between a baseline value of the threshold parameter and a threshold value of the threshold parameter; wherein the baseline value is calculated using the baseline ECG data, and the threshold value is used to define a predetermined criterion.

According to some embodiments of the present disclosure, the baseline ECG data are received from the patient before the intervention procedure.

According to some embodiments of the present disclosure, the transcatheter intervention procedure includes an action that might induce the heart conduction block.

According to some embodiments of the present disclosure, the baseline ECG data are received from the patient during the transcatheter intervention procedure and before the action that might induce the heart conduction block is taken.

According to some embodiments of the present disclosure, evaluating the ongoing ECG data to obtain an indication of sub-threshold heart conduction block includes combining the range parameters into a single range parameter having a minimum value when each of the range parameters has a value equal to its baseline value, and a maximum value when the predetermined criteria indicate the existence of the heart conduction block.

According to some embodiments of the present disclosure, evaluating the ongoing ECG data to obtain an indication of sub-threshold heart conduction block includes combining the range parameters into a single range parameter having a maximum value when each of the range parameters has a value equal to its baseline value, and a minimum value when the predetermined criteria indicate the existence of the heart conduction block.

According to some embodiments of the present disclosure, the indication is an indication to a heart conduction block at a specific structure of the heart conduction system.

According to some embodiments of the present disclosure, presenting the indication includes marking a visual representation of a heart conduction system with a mark presented at the specific structure of the heart conduction system and having a visual characteristic indicative to the indication.

According to some embodiments of the present disclosure, the predetermined criteria refer to leads of a standard 12-lead ECG.

According to some embodiments of the present disclosure, the ongoing ECG data are obtained using a set of electrodes, having less electrodes than defined for performing a standard 12-lead ECG.

According to some embodiments of the present disclosure, the set of electrodes includes four electrodes.

According to some embodiments of the present disclosure, baseline ECG data were collected using a standard 12-lead ECG.

According to some embodiments of the present disclosure, the four electrodes correspond to the arm and leg electrodes of the standard 12-lead ECG, and one of the chest electrodes.

According to some embodiments of the present disclosure, the chest electrode is the V6 electrode.

According to some embodiments of the present disclosure, the evaluating includes transforming the ongoing ECG data to standard ECG data referring to leads corresponding to a standard 12-lead ECG.

According to some embodiments of the present disclosure, transforming the ongoing ECG data to standard ECG data includes performing a modified inverse Dower transform to produce a Frank set of ECG data corresponding to Frank vector cardiography leads, and performing a Dower transform on the Frank set of ECG data to yield the leads corresponding to a standard 12-lead ECG.

According to an aspect of some embodiments of the present disclosure, there is provided a device for intraoperative monitoring of new onset conduction disturbances, the device including:

an indicator device configured to present an alarm responsive to alarm instruction; a processor coupled to the indicator device; and a digital memory accessible to the processor and storing criteria for determining existence of a heart conduction block based on ECG data; wherein the memory also stores instructions instructing the processor to: access a portion of an ongoing ECG signal recording recorded from a patient during the performance of a transcatheter heart procedure; calculate a test value of a metric indicative of a level of partial heart conduction block from the portion of the ongoing ECG recording using baseline ECG data recorded from the patient and the stored criteria; and send an alarm instruction to the indicator device based on the calculated test value.

According to some embodiments of the present disclosure, together with ECG circuitry is configured to record the ECG signal recording.

According to some embodiments of the present disclosure, the digital memory stores the baseline ECG data.

According to some embodiments of the present disclosure, the instructions instruct the processor to: transform threshold parameters used in the criteria into range parameters; and calculate the test value based on the range parameters.

According to some embodiments of the present disclosure, the instructions instruct the processor to transform a threshold parameter used in the criteria to a range parameter by establishing a range between a baseline value and a threshold value, wherein the threshold value is used in the criteria and the baseline value is derived from the baseline ECG data using the rules.

According to some embodiments of the present disclosure, the stored criteria are for determining existence of a heart conduction block in a heart of any patient based on ECG data.

According to some embodiments of the present disclosure, the instructions instruct the processor to calculate the test value by combining the range parameters into a single range parameter having: a minimum value when each of the range parameters has a value equal to its baseline value, and a maximum value when the predetermined criteria indicate the existence of the heart conduction block.

According to some embodiments of the present disclosure, the instructions instruct the processor to calculate the test value by combining the range parameters into a single range parameter having: a maximum value when each of the range parameters has a value equal to its baseline value, and a minimum value when the predetermined criteria indicate the existence of the heart conduction block.

According to some embodiments of the present disclosure, the alarm instruction is indicative of heart conduction block at a specific structure of the heart conduction system.

According to some embodiments of the present disclosure, the indicator device displays a visual representation of a heart conduction system with a mark presented at the specific structure of the heart conduction system and with a visual characteristic indicative of the alarm instruction.

According to some embodiments of the present disclosure, the stored predetermined refer rules to leads of a standard 12-lead ECG.

According to some embodiments of the present disclosure, the ongoing ECG signal recording is obtained using a set of electrodes having less electrodes than defined for performing a standard 12-lead ECG, and the device includes an interface connecting to electrodes with fewer electrode attachment ports than ten.

According to some embodiments of the present disclosure, the set of electrodes consists of four electrodes.

According to some embodiments of the present disclosure, the digital memory stores the baseline ECG data, collected using standard 12-lead ECG.

According to some embodiments of the present disclosure, the four electrodes correspond to the arm and leg electrodes of the standard 12-lead ECG, and one of the chest electrodes.

According to some embodiments of the present disclosure, the chest electrode is the V6 electrode.

According to some embodiments of the present disclosure, the instructions instruct the processor to transform the ongoing ECG data recorded from the patient into a standard ECG data referring to leads corresponding to a standard 12-lead ECG.

According to some embodiments of the present disclosure, the processor transforms the ongoing ECG data by performing a modified inverse Dower transform to produce a set of leads corresponding to Frank vector cardiography leads, and a Dower transform on the produced set of leads to yield the 12-lead ECG.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A schematically represents an alarm system configured to signal the detection of electrocardiogram signals potentially associated with heart conduction system injury, according to some embodiments of the present disclosure;

FIG. 1B, which is a block diagram schematically representing components of an alarm system configured to signal the detection of electrocardiogram signal changes potentially associated with heart conduction system injury, according to some embodiments of the present disclosure;

FIG. 2 schematically represents an algorithm configured for detection of changes in the relative timing of certain events present within a recorded body surface ECG signal, according to some embodiments of the present disclosure.

FIG. 3 schematically represents features of a single body surface ECG cycle, according to some embodiments of the present disclosure;

FIG. 4 schematically illustrates elements of a threshold-style alarm system, according to some embodiments of the present disclosure;

FIG. 5 schematically illustrates a graph (in two dimensions) of baseline and potentially alarm-triggering vector electrograms, according to some embodiments of the present disclosure;

FIG. 6 schematically outlines a method of setting alarm levels for the device of FIGS. 1A-B, according to some embodiments of the present disclosure;

FIG. 7A schematically illustrates anatomical elements of the conduction system of a heart;

FIG. 7B schematically illustrates user interface elements for indicating existing and/or incipient heart block, and adjusting heart block alarm settings, according to some embodiments of the present disclosure;

FIG. 8 schematically illustrates algorithmic strategies used to convert clinical criteria for block and/or partial block into algorithms for graded assessment of sub-threshold block, and use of those algorithm, according to some embodiments of the present disclosure; and

FIG. 9 is flow chart schematically illustrating a method of generating a synthetic-lead ECG from a reduced set of measurement electrodes, according to some embodiments of the present disclosure.

### DESCRIPTION OF SPECIFIC EMBODIMENTS OF THE INVENTION

The present invention, in some embodiments thereof, relates to the field of transcatheter surgeries and more particularly, but not exclusively, to structural heart disease interventions.

### Overview

A broad aspect of some embodiments of the present disclosure relates to the potential reduction and/or mitigation of complications and adverse outcomes from treatments affecting heart structure; for example, structural heart disease (SHD) interventions, according to some embodiments of the present disclosure. Complications of particular concern in some embodiments of the present disclosure are those which result from damage to structures of the heart's conduction system; for example, the bundles of His and the atrial-ventricular node. Any heart intervention which manipulates tissue in the vicinity of these structures is a potential target of some embodiments of the present disclosure, for example, heart ablation treatments and/or valve repair treatments.

Structural heart disease (SHD) refers to all non-coronary cardiac diseases that impair normal cardiac function and are associated with an increased risk of heart failure and death. They include congenital heart defects and acquired defects related to aging, injury, or infection (e.g., as in valve dysfunction). Common structural heart defects treated with percutaneous therapies include valvular heart diseases such as aortic stenosis, patent foramen ovale (PFO), atrial septal defect, ventricular septal defect, left ventricular aneurysm, patent ductus arteriosus, paravalvular leak, and hypertrophic cardiomyopathy. Structural interventions may be performed to treat other diseases; for example, closure of the left atrial appendage in patients with atrial fibrillation.

The burden of valvular heart disease in a growing population of individuals above the age of 75 is considerable with more than 1 in 8 people having moderate or severe atherosclerosis (AS). Aortic valve stenosis is a type of SHD and is the most common cause of left ventricular outflow obstruction in children and adults. Valvular AS can lead to progressive narrowing of the aortic valve orifice that may result in endocarditis, embolic events, arrhythmias, heart failure and sudden death. In adult patients with symptomatic severe AS who do not undergo valve replacement, nearly 75% will not be alive at 5 years following symptom onset.

Improved pre-procedural planning, device innovations, operator experience, and evolution of implantation techniques have reduced the incidence of serious peri-procedural complications including death and bleeding. However, despite these advancements, a range of complications limit the efficacy of commonly performed structural heart disease (SHD) interventions, such as (transcatheter aortic valve replacement) TAVR and (left atrial appendage occlusion) LAAO and constitute unmet needs. In particular, certain SHD interventions, for example TAVR, may comprise a risk for new onset conduction disturbances (NOCDs) including one or more of left bundle branch block (LBBB), right bundle branch block (RBBB) and atrioventricular block (AVB). Such blocks may, for example, require permanent pacemaker (PPM) implantation following a procedure such as TAVR.

The occurrence of NOCDs including high-grade atrioventricular block (AVB) requiring PPM implantation and new-onset LBBB remain a common complication following TAVR. At present, the reported incidence of NOCD after TAVR varies widely ranging from 5-65% in studies using various types of transcatheter heart valves (THVs). A meta-analysis of 12 TAVR studies found that new onset LBBB occurred in 22.7% of patients and PPM implantation was reported in 6% to 32% of patients, and that both types of NOCD were associated with a 1.3 fold increased risk of all-cause death (19.8% vs. 15%, p<0.001) and 1.35 fold increased risk of heart failure hospitalization (7.2% vs. 5.8%, p<0.001) at 1-year follow-up. PPM implantation following TAVR is not only associated with increased risk of death and heart failure, but is associated with a reduction in quality of life and increased cost.

Many other SHD interventions including mitral and tricuspid repair or replacement can cause NOCD, similarly SHD interventions of the inter atria or inter ventricular septa are associated with NOCD. Heart ablation, if inadvertently performed near critical features of the heart's conduction system, may also result in NOCD.

Without commitment to a particular theory of causes, it appears that SHD interventions may particularly result in NOCD through various types of mechanical insult that may occur as tissue is manipulated, and/or as devices are implanted. Pressure on tissue can come from pre-implantation activities such as balloon expansions, from implantation activities that involve expansions of devices (e.g., replacement valves), from devices which anchor by pinching tissue (e.g., septal repair devices) and/or from penetrating tissue (e.g., annuloplasty devices). Pressure can squeeze tissue. Pressure can change how hard structures such as accumulated calcifications impinge upon vulnerable structures. Anatomical variation may play a role in NOCD—for example, a heart conduction system structure may be more or less exposed to damage, depending on anatomical variation specific to the patient. Anatomical variation may also change where the “safe position” is for an implanted device, or even whether there is such a safe position.

In addition to any background risk of working in the vicinity of potentially sensitive conduction structure, there may be particular actions or situations during an SHD intervention for which the risk of acutely inducing NOCD is especially high; for example, at points where tissue is being forcibly expanded, and/or a device is forcibly advanced or anchored.

An aspect of some embodiments of the present disclosure relates to providing intraoperative warnings of existing, developing, and/or potential new onset conduction disturbances (NOCD) based on ECG recording data, according to some embodiments of the present disclosure. In some embodiments, the operating procedure comprises implantation of a device to treat structural heart disease (SHD); for example, a replacement heart valve, leaflet coaptation device, annuloplasty device, and/or septal defect repair device. In some embodiments, the operating procedure comprises manipulation of tissue to repair SHD.

In the case of certain surgical procedures, e.g., annuloplasty and/or valve implantations, there is a moderate to high risk of inadvertent insult and/or injury to electrical transmission pathways of the heart conduction system. One way in which this potentially manifests is as complete or partial heart conduction block—a slowing and potentially complete prevention of signal transmission along damaged pathways.

Slowing of transmission along these pathways tends, in general, to affect recordings of body surface ECG. One way to see such effects is to attend to the amplitudes and/or timings of landmark ECG features (e.g., onsets, offsets, and/or peaks) defining the P wave, QRS complex, and T wave. Slowed transmission may, for example, extend the P-to-QRS interval, and/or affect the width of the QRS waveform.

As a result of insult or injury (temporary or permanent) which impairs the functioning of the heart conduction system, the magnitude and/or relative timing of these landmark features may change. Of particular interest, in some embodiments of the present disclosure, is the duration of period measured between a landmark feature of the P wave (e.g., its offset), and a landmark of the QRS wave (e.g., Q wave peak). During this interval, electrical transmission of the heart depolarization wave travels from the atrium along and/or to certain critical features of the heart's conduction system and into the ventricle. These critical features include, for example, the sinoatrial (SA) node, the atrioventricular (AV) node, the bundle of His (comprising left and right bundles), the Purkinje fibers, and finally the contractile ventricular epicardium of the two ventricles. The relative timings, amplitudes, and even directionality of the various waves making up a standard ECG signal depends to some degree on the rate of transmission along these pathways, as well as recording positions used to generate the ECG lead being examined.

Heart conduction block may itself be “sub-threshold” in nature—that, is, a reduction in function from normal conduction subtle enough that standard clinical diagnostic criteria would miss it, or treat as ambiguous. Herein, the term “sub-threshold” is applied to heart conduction block which is clinically sub-threshold in this sense. In particular, the term may be understood to distinguish from types of “partial heart conduction block” which are clinically significant according to standard diagnostic criteria; e.g., it is possible to have heart conduction block which is “sub-threshold” even compared to clinically recognized “partial” heart conduction block, insofar as there exists a range of partially reduced heart conduction function in between “no block at all” and clinically recognized “partial” heart block.

Furthermore, given normal patient state and patient population variability, it may be understood that clinical criteria for diagnosing various states of heart block are selected to strike a suitable balance of false positives and false negatives across a wide patient population. One potential result of this is that below the clinical threshold which clinical criteria establish there is nonetheless a range of sub-threshold heart conduction blocks (different from a completely unblocked condition) that the general patient population criteria do not distinguish. Close monitoring during a procedure which might damage heart function, however, provides an opportunity to detect changes in heart block condition which may be rapid enough and/or large enough to warrant alerting the physician, even though they would not necessarily be significant for a member of the general population.

In particular, although the ECG signal is subject to natural variability over time in any case, some ECG signal changes may be sufficiently distinctive as to warrant interrupting a physician with an alert or warning that potential NOCD has been detected. These ECG changes may sometimes be distinctive by themselves, and in other cases distinctive only when arise during an intervention, e.g., in comparison to pre-intervention ECG signals.

In some instances, the conditions of an SHD treatment may lead to ECG changes which are, for example:
Sudden: e.g., readily isolated by comparison with recently recorded (within the past 10 seconds) signals.
Locale-particular: i.e., the change is characteristic of damage/insult at a working. location of treatment procedures—raising the suspicion that the work is what caused the change.
Correlated to treatment actions: i.e., arising within a time window of seconds or minutes following an action which could affect heart conduction.

Accounting for these situational conditions may help to refine the detection of signal changes which might otherwise be considered insignificant or uninterpretable.

In some embodiments, the clinical criteria chosen are derived from and/or tuned (modified) to apply to a more particular patient population, selected to be more like the patient actually undergoing the procedure than the general population as a whole. For example, the more particular population may be a population of patients who are actually candidates (and/or near-candidates) for the same procedure the patient is undergoing, and/or who have previously undergone the procedure. ECG recordings from this patient population are optionally used as the data based upon which the clinical criteria are derived and/or modified. Optionally, this more particular population also includes patients (and their ECG data) for whom some degree of change in heart block condition was actually observed: for example, during their own procedure; or subsequently, whether or not the subsequent change correlated with their own procedure. Optionally, the clinical criteria are derived and/or tuned based on observations of what changes in ECG signals are empirically found to be indicative of (e.g., correlated with) heart block for the more particular population. Such clinical criteria derived from the more particular population and their ECG data have the potential advantage of better defining the range of what is considered acceptable within the context of a procedure—given that the patient is already ill—potentially reducing false positive results leading to an alarm, without unduly reducing alarm sensitivity.

It may be noted that where a sufficient number of patients of the more particular population are available, the criteria may be developed based on one or more machine learning algorithms. Outputs of the one or more algorithms may themselves be probabilistic (weighted) assessments of whether a given present monitored condition of a patient is or is not in a state of heart block; or the machine learned outputs may be binary, and interpolation used between pre- and post-block states and/or data to generate examples and/or weighted assessments of patient conditions having intermediate levels of heart block, heart block likelihood, and/or heart block risk.

In some embodiments, ECG monitoring is carried out for a period of time that includes a time without heart conduction delays due to new onset conduction block (e.g., before the procedure begins), and a time (e.g., during the procedure) potentially with heart conduction delays due to new onset conduction block. Optionally, ECG signal recordings from the period without new onset conduction block (e.g., before the initiation of treatment activities associated with NOCD risk) are used to define a baseline condition (reference value) against which later ECG recording periods are compared.

ECG signal recordings may trigger an alarm during the treatment procedure itself, or during a post-operative period of monitoring.

Comparisons to determine the presence of NOCD may use new deviations from a normal baseline (e.g., a new difference of a test value from a reference value), and/or new similarity to an abnormal reference value (e.g., a new matching of a test value to within a range of reference values indicative of NOCD).

An aspect of some embodiments of the present disclosure relates to the use of a database which collects information indicative of values of the non-ECG factors and the corresponding occurrence of heart conduction block co-associated with instances of transcatheter heart procedures.

In some embodiments, co-association in the database of values of the non-ECG factors and the occurrence of heart conduction block is used to set comparison criteria which help determine whether a value of a metric, calculated from a portion of an ongoing ECG for a current procedure, does or does not trigger an alarm indication.

For example, if the values of non-ECG factors of the patient of the current procedure are similar to the values of non-ECG factors of other patients for whom an elevated risk of heart conduction block has been recorded, then the current patient may also be considered to be at elevated risk.

The finding of elevated risk may provide a reason to modulate the alarm settings; e.g., to trigger the alarm in response to ambiguously small changes, in the interests of caution.

Herein, the term “metric” refers to a measure numerically indicative of some property. For example, Euclidean distance is metric of the property of distance which is expressed by the equation d=\sqrt{x²+y²+ . . .}. Rectilinear distance is another metric of the property of distance expressed by the equation d=|x₁−x₂|+y₁−y₂|+ . . . . Particular metrics may be used in the detailed specification of parameters; for example, a parameter of amplitude may be expressed by the selection of a metric defining how the peak and base of the amplitude are selected. Use of the term metric also emphasizes the distinction of a measurement from the property it associates to. The property of “peak width” of a signal portion, for example, is plausibly measured with equal validity by a plurality of metrics, and there is no contradiction in the same peak's width being given different measurement values by different metrics applied to it. “Metric” is not used herein in the sense of the metric system for the standardized measure of weights, distances, times, and other properties, unless explicitly stated otherwise.

An aspect of some embodiments of the present disclosure relates to the automatic detection and warning of NOCD at its time of onset, during a critical period during or following a procedure which carries a notable risk of creating NOCD.

It should be noted, in this regard, that an NOCD might be treated as lacking clinical significance (sub-threshold according to standard diagnostic criteria) when occurring “naturally”, although still considered to be beyond a threshold deserving of an alarm if found to occur as a result of an operative procedure.

There are at least two reasons for the difference. First, since the cause of the NOCD is known or strongly suspected, there remains a potential for reversing the conduction disorder (perhaps especially a weak one), e.g., by removing, replacing, and/or repositioning a device which is apparently responsible for the change. Second, a weak NOCD appearing early could be a harbinger of a stronger (e.g., clinical-level) conduction disorder which could develop over time.

In some embodiments, ongoing ECG monitoring is begun before, at the time of, or soon after the performance of a surgical procedure (e.g., a transcatheter SHD treatment procedure) which carries with it a risk of creating NOCD.

If, during the monitoring, the ECG transitions to contain indications of NOCD (e.g., undergoes changes that are indicative of conduction block, such as increases in certain intervals in the ECG signal), then an alarm is delivered to alert a human medical caretaker. The caretaker may be the surgeon performing the treatment (e.g., if the treatment is still underway), and/or, post-operatively, a responding attendant—who may, for example, be located out of the presence of the patient (e.g., in another room) at the time of the alarm. Presentation of the alarm is substantially immediate after detection, e.g., a message is sent and/or an alarm device is activated within a second of the detection.

In some embodiments, the alert is configured to carry with it an indication of the time of NOCD onset. Optionally the time is indicated more particularly with respect to one or more earlier events (such as the treatment procedure overall, or some step of the treatment) which are concerns for elevating the risk of NOCD. The time information is potentially helpful in assessing mitigation strategies, and/or identifying the treatment action and/or parameter responsible for the NOCD alarm. The NOCD alarm may, for example, be due to a gradual pressure-induced decline in conduction. Detecting the gradual decline early enough may allow successful remedial action, e.g., to remove or reposition an implanted device, potentially halting or reversing the NOCD.

In some embodiments, the alert is configured to carry with it an indication of the severity of the NOCD onset. For example, the alert may convey whether the identification of NOCD onset is tentative (for example, still in a state that crosses and recrosses a threshold of detection) or severe (e.g., crossing and exceeding a detection threshold); and/or gradual (for example, drifting slowly but detectably over a period of several hours from a baseline value) or sudden (e.g., appearing within seconds or minutes).

In some embodiments, the detecting performed during the monitoring is tuned based on parameters particular to the patient and/or the treatment procedure which the patient has undergone. For example, a patient associated with factors of elevated risk may be monitored using settings chosen with relatively greater concern for avoiding false negatives as opposed to false positives.

An aspect of some embodiments of the present disclosure relates to indicating that a patient is approaching NOCD, while their actual conduction disturbance is still sub-threshold with respect to standard diagnostic criteria. In some embodiments, the indication of approaching NOCD is derived from a transformation of standard groups of clinical criteria into calculations which provide graded metrics indicative of the functional status of structures of the heart conduction system.

Clinical criteria groups may be structured to generate distinct categories, e.g., via use of a decision tree, as befits their origin in support of clinical decision making—e.g., diagnosis of a disease state being in or out of a certain category, and/or supporting a decision to do or not do something, such as perform a treatment intervention. It is an object of some embodiments of the present disclosure to provide indications that a patient is approaching a clinically significant state while their actual state is still sub-threshold, according to a formal or informal categorizing framework that a given criteria group establishes.

Clinical assessment criteria for heart conduction block may be expressed in terms of, for example:
The absence or presence of certain features in measured ECG signals.
The relative values of certain ECG signal features (comparisons of amplitudes and/or durations, for example).
Thresholds (e.g., of intervals and/or amplitudes of features in measured ECG signals).
Auxiliary features (e.g., “typical” but not required features, or features which negate the result of some other criterion when present).

The criteria may play one or more roles within the criteria group, for example:
To establish a basic finding that some condition of potential medical significance is present.
To help detect a feature of a particular condition which the criteria group is designed to identify.
To rule out other conditions. For example, a slowdown in some measure of heart conduction might be due to damage at one of a plurality of branches of the heart conduction system. A criterion additional to the slowdown itself may be introduced to help rule out one or more of the branches.
To establish validity; for example, to avoid generating a mistaken finding of a heart block when some other overriding condition such as fibrillation is present that invalidates assumptions behind the application of other criteria.

An object of some embodiments of the present invention is to yield values, which indicate graded sub-threshold progress toward clinical criteria. In some embodiments, such values are obtained based on criteria arranged to yield categorical conclusions; so that such embodiments include converting categorical criteria into graded criteria. The original, categorical, criteria may be arranged in a formal logical structure.

By “categorical” it is mean that the outputs of the criteria are in the form of categories such as “no block”, “LBBB” (left branch bundle block) or “possible LBBB” (possible left branch bundle block). Even though one of the categories is indefinite, it is still “categorical” in the sense that it does not indicate how close to the threshold of full “LBBB” the various tests of parameter values come. Either the category applies, or it does not.

In some embodiments, conversion of a categorical criterion to a graded range criterion (referred to herein as defining a “range parameter”) includes the generation of one or more parameter ranges corresponding to respective original one or more clinical criteria, each expressed in terms of a threshold. For example, the original categorical parameter may be in the form of a conditional statement such as “if QRS area in V1<−100 mVms”, where −100 mVms is the threshold value. Such criteria are also referred to herein as defining a “threshold parameter”.

The graded parameter range (also referred to herein as a “range parameter”, in some embodiments, is set using a baseline value for the respective parameter, and the value for the threshold parameter obtained from the clinical criteria.

The baseline value, in some embodiments, is obtained by measurements from the patient. For example (continuing the previous example), the patient may have an initial baseline value of QRS area in V1=−50 mVms. The baseline value may be measured and/or selected. In particularly, the baseline value may be personalized to an individual patient, e.g., because it is itself based on measurements of an ECG of the patient, and/or selected based on factors in the known clinical history of the patient (e.g., a known pre-existing level of sub-threshold heart conduction block).

The baseline and threshold values are optionally used to generate a normalized scale, e.g., a scale ranging between 0 and 1. In the example being used, the scale range 0-1 is mapped to the measurement range of −50 mVms to −100 mVms.

Logical operators such as AND and OR appearing in the original categorical rules may be substituted in the corresponding graded rule by other operators, such as minimum value selection (e.g., for AND), maximum value selection (e.g., for OR), or another function. For example, the compound (comprising more than one sub-expression) criterion “QRS area in V1<−100 mVms and S amplitude in V6<=1000 μV” is converted to corresponding normalized ranges (e.g., between 0 and 1). Using minimum value selection, the AND operation is converted into selection of the lesser of the current values for the two normalized ranges.

Criteria expressed in qualitative terms may also be substituted by graded rules. For example, a compound clinical criterion (itself part of a larger compound clinical criterion, or “group of criteria”) may be expressed as “broad notched or slurred R wave in leads I, aVL, V5, and V6 and an occasional RS pattern in V5 and V6 attributed to displaced transition of QRS complex.” According to the informally expressed logic of the subgroup sentence, the RS pattern is “occasional” along with the notched/slurred R wave. Accordingly, it is not used as a stand-alone alternative in its own right: it is neither required (AND-like), nor sufficient (OR-like). In some embodiments of the present disclosure, such criteria are incorporated into a graded output using selected functions or function-adjusting coefficients, e.g., used to adjust connected criteria through a non-linear function such an exponential or sigmoid function. Herein, such criteria incorporated in this fashion are also referred to as “intensifiers”. Such criteria can be positive intensifiers (increasing the significance of one or more co-criteria), negative intensifiers (decreasing the significance of one or more co-criteria), or both (in a manner depending on their own ranged value). The effect of such functions can be, for example, to raise and/or lower the value of some other evaluated criterion. In the given example, the ranged definition or definitions attached to “broad notched or slurred R wave” produce a value within the range of 0-1. The presence of the “RS pattern” can be used, e.g., to move that value closer to 1, with the rough meaning that an intermediate indication of a broad notched or slurred R wave is considered more significant when an RS pattern is also noted. Additionally or alternatively, the absence of the RS pattern can be used to move that value closer to 0, with the rough meaning that an intermediate indication of a broad notched or slurred R wave is considered less significant when an RS pattern is not noted.

An aspect of some embodiments of the present disclosure relates to the conversion of clinical definitions categorizing states of heart block into a monitoring regime suitable for the on-line monitoring of patient heart state as it may trend towards one of the categorized states, while still remaining outside of the clinical definition of the state itself.

In some embodiments, the clinical criteria used include the MEANS (Modular ECG Analysis System) criteria developed by the Department of Medical Informatics at the Erasmus University of Rotterdam in the Netherlands (Bemmel J H et al., “Methodology of the modular ECG analysis system MEANS”, Methods Inf Med. 1990 September;29(4):346-53), and/or other published criteria, e.g., as described by Surawicz et al. in “AHA/ACCF/HRRS Recommendations for the Standardization and Interpretation of the Electrocardiogram” (J. Amer. College of Cardio., 53:11, 2009 976-981).

The MEANS system in particular has been developed for use in computerized ECG analysis to yield diagnostic classifications. Evaluation of this system has been described, for example, by Willems J et al. in “A reference database for multi-lead electrocardiographic computer measurement programs”. (J Am Coll Cardiol 1987;10:1313-21) and Willems J L et al. in “The diagnostic performance of computer programs for the interpretation of electrocardiograms” (N Engl J Med 1991;325:1767-73). Evaluations described in these references comprised comparison of human referee evaluations of ECG data with computerized evaluations of ECG data, from cases where the ECG data itself was selected for showing certain abnormalities and/or cases which were clinically validated instances of various clinical disorders. These examples show how not only the clinical criteria, but also the ground truth used to help establish and evaluate them may be categorical in nature: ECG data used are from patients already in particular clinically defined states, and it is with respect to these states that the classification criteria are evaluated (and if necessary, modified during their initial development).

In some embodiments of the present disclosure, an aim of heart block monitoring is to watch for signs of patient state transition between a “no block” state, in which the ECG is not indicative to a heart block of a certain category, and a “block” state, in which the ECG is indicative to the existence of the heart block of the certain category. While in the prior art such a transition is stepwise, in some embodiments, the monitoring is aimed at watching as this transition takes place gradually, and raise an alert upon sensing that the patient reached some intermediate point between the “no block” and “block” states. Accordingly, in some embodiments, partial transition towards a block state is detected, while the prior art is blind to such partial transitions.

Under ordinary circumstances, transitions from “no block” to “block” state are, for example, too gradual or too unexpected to be practically detected by use of ECG monitoring. Some surgical situations, however, carry with them a risk of acutely damaging and/or impairing the function of structures of the heart conduction system, making the option of monitoring for signs of state transitions in real time (i.e., as they occur) potentially practical.

The inventors found that detecting such state transitions pose some problems. One problem is that the categorical definitions of heart block are multivariate (defined by a plurality of parameters). Accordingly, monitoring is sometimes not just a matter of monitoring progress of a single parameter toward a category-defining threshold; there may be several thresholds to account for which define the category in combination. In other words, multivariate state data need to be somehow processed to funnel them into a decision to raise an alert to the development of clinically sub-threshold heart block. Optionally, the decision itself is based on the value of a single metric, variable over a range of values. In some manner, exemplified below, that range is constructed using clinical criteria which originally generate only categorical assessments of heart block state (e.g., “in” the state or “out” of the state).

Insofar as the clinical criteria are categorical, data indicating how measurements of heart function evolve on the way to a new heart block state are potentially unavailable. So if there are, e.g., two ECG event intervals (e.g., P-R interval and R-T interval) which must each cross a certain threshold of length to indicate a certain state of heart block, it may not be clear from the clinical criteria whether they should be assumed to each change proportionally toward the state, whether one interval's lengthening leads that of the other, or whether some other pattern typically holds. Potentially, there is no single typical pattern.

In some embodiments of the present disclosure, this limitation is bypassed by using a combining approach based largely on the implied or explicit logical structure of the categorical set of clinical criteria. In some embodiments of the present disclosure, a combining approach that is used comprises converting logical operations such as AND and OR into comparison operations such as maximum and minimum operations.

Additionally or alternatively, other types of operations are used; for example, functions which allow parameters to influence each other asymptotically, e.g., via a sigmoidal co-function that allows one parameter to influence the weighting of another, with the effect that it can be treated as being “closer to” or “further from” the threshold value that its standalone value might indicate. These other types of operations may be useful in particular when the clinical criteria are expressed informally—e.g., using indefinite phrases such as “often with”, “occasionally with”, “seldom with”, “or sometimes”, etc.

Using the clinical criteria's own logical structure to generate a ranges metric of heart block state generally has the advantage of being applicable to discern movement of a patient's heart block state toward a new categorically defined state, even in the absence of a predetermined map of how the various parameters that make up the multivariate state co-evolve. In particular, for purposes of raising an alarm in response to intra-surgical adverse events, it is potentially less important to know exactly how far a patient has progressed toward a heart block state, than it is to know that a detectably large (and concerning) change has occurred and/or is underway.

Related to the definition of “large change” there is an additional problem of how to set the range through which heart block state may be observed to evolve during monitoring. In some embodiments of the present disclosure, the upper limit of the range (e.g., a value normalized to 1=“block”) is set to be a state where the set of clinical criteria that define the clinical state of heart block are jointly met. Typically, this is the boundary at which all thresholds of the clinical criteria are met. Although there may be changes continuing beyond this limit, whatever alert is provided for is preferably raised at least by the time that the full clinical state definition is met, and preferably earlier.

As to the lower limit, it is preferably defined to be low enough to be able to show sub-threshold movements toward a new clinical state (e.g., if it was the same as the range's upper value, there would be no detection of sub-threshold changes in parameters). However, the lower range value is also preferably defined to be high enough to avoid a meaningless “dead zone,” where the patient is not expected to be. More particularly, different patients may preoperatively already be “different amounts of the way” toward the state defined by the clinical criteria. In some embodiments, the lower end of the range is selected to be defined by an actual pre-existing state (prior condition) of the patient which is to be monitored.

The prior condition can be, for example, a completely healthy state, or an intermediate state between “no block” and “block”. In some embodiments, the prior condition may be defined separately for each possible block. For example, the prior condition of a patient may be determined to be free of any block in the bundle of His, but on the way towards a block of the upper left ventricle.

For example, two patients may be in different prior conditions in respect to a “right bundle branch block” (RBBB). The first patient prior condition is partially on the way towards developing a RBBB (e.g., parameters such as ECG event intervals are increased due to reduced conduction velocity), while the second patient's prior condition is normal, at least as far as parameters indicative of right bundle branch conduction show. In both patient it may be prudent to alert the doctor when the patient advances further toward RBBB, but in the second patient more advancement may be allowed before alert, than in the first patient.

It may also be noted that per-patient tailoring of the monitoring range creates a customized monitoring regime, which is nonetheless derived from heart block criteria defined for a more general population.

An aspect of some embodiments of the present disclosure relates to re-expressing (“expanding”) ECG recordings made using a reduced subset of electrodes as if they were made using a system of lead definitions (for example, the standard 12-lead ECG system) defined with respect to electrodes not actually used. The expansion transforms data recorded by a reduced subset of ECG electrodes into a form that may be analyzed by analysis tools designed for analyzing inputs provided in terms, e.g., of the standard 12-lead system. Such analysis tools may include, for example, a group of clinical criteria (i.e., one or more compound criteria) for conduction block identification.

In some embodiments, clinical criteria are expressed in terms of the leads of a standard 12-lead system, but used to identify clinical condition based on data recorded without some of the electrodes that the standard 12-lead system assumes. For example, only 3-6 electrodes may be used out of the 10 standard electrodes of the 12-lead system. Such configurations are also referred to herein as “reduced-electrode” ECG systems. In some embodiments, the lead subset actually available is expanded to the 12-lead set by an intermediate transformation to the Frank lead system (used in vector cardiography) via a modified inverse Dower transform, and then to the 12-lead system via a Dower transform. The expansion to 12 leads does not create new information (e.g., data associated with different leads of the expanded 12-lead system may be more highly correlated with each other than would be seen in a true 12-lead ECG), but the remapping has potential advantages for use in the generation of graded criteria (e.g., useful for detecting clinically sub-threshold changes in parameters used to assess heart block) from groups of clinical criteria that make explicit reference to the leads of a 12-lead ECG.

It should be noted that the transformation also optionally allows mixed use of ECG data obtained using the 12-lead system with ECG obtained using a reduced-electrode system. For example, baseline data may be obtained using the 10 electrodes defined for a 12-lead system, and intraoperative monitoring ECG data obtained using 3-6 electrodes, for example, 3 electrodes or 4 electrodes.

In some embodiments, data recorded using a reduced-electrode system are corrected based on comparison between them and data recorded using the complete ECG leads; for example, when the patient was in rest, before the operation. Data from complete ECG leads may be generated for a baseline recording using a true 12-lead system (e.g., using 10 recording electrodes) and leads generated by transformation to a 12-lead system using a subset of the same electrodes used in recording the 12-lead system. The subset is preferably selected to correspond to the arrangement of electrodes in the reduced-electrode ECG recording system which is used intra-procedurally.

In some embodiments, corrections are made to the transformation to 12 leads of the reduced-electrode ECG recording system used intra-procedurally, based on the comparison. In effect, this provides a way to estimate some of the information “missing” from the reduced-electrode ECG recording system. In some embodiments, the correction comprises comparing same baseline metrics (e.g., event intervals) for the true 12-lead system and the 12-lead system transformation of electrode subset recordings. Where metrics differ, a correction is optionally generated to make them match again. A correction may be an offset in milliseconds, for example. Applied to intra-procedurally recordings using a reduced-electrode ECG system, the correction potentially helps to maintain correspondence between metrics calculated from transformed ECG system leads with metrics that would have been calculated if the original recording ECG system was a true (e.g., 10 electrode) 12-lead system. Other correction types, for example, linear corrections, may be generated using reference data similar to that used to develop and/or test the categorical criteria. For example, 12-lead ECG data obtained from patients in various states of clinically verified heart block can be compared to 12-lead ECG data transformed from reduced-electrode subsets of the same ECG data. In cases where the correction needed to match them is different from the correction determined from the baseline data, a linear (or otherwise interpolated) correction can be generated that makes the applied correction different as a function of parametric distance between baseline state (e.g., of the individual patient) and clinical heart block state (e.g., as determined from a population of subjects).

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### ECG-Based Alarm System, and Methods of Operation

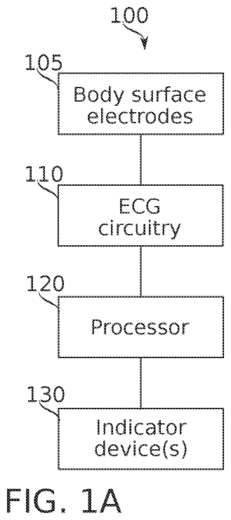

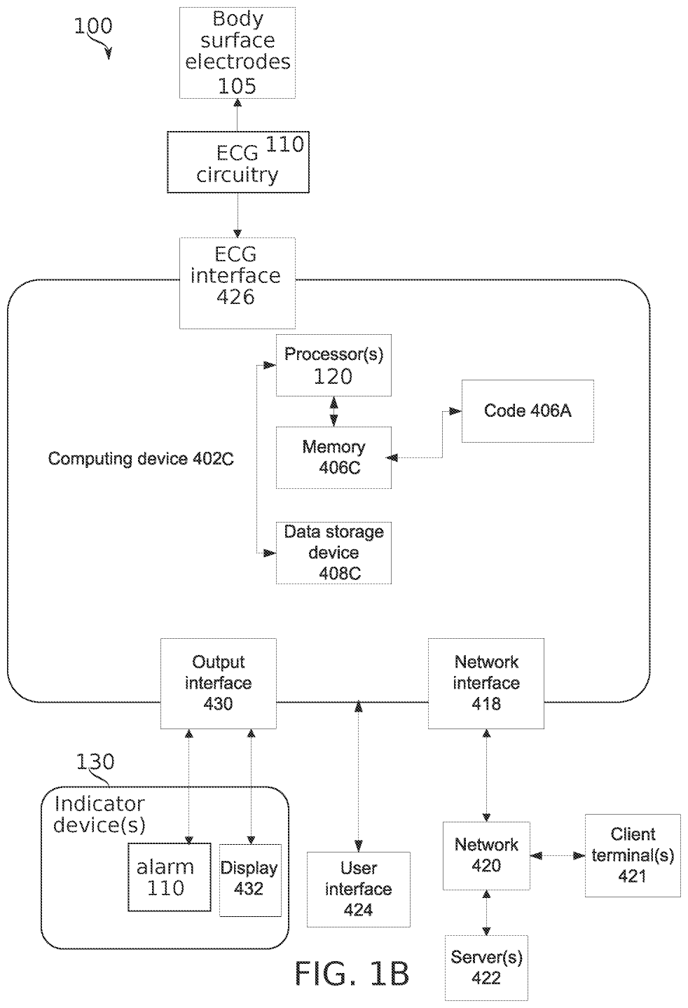

Reference is now made to FIG. 1A, which schematically represents an alarm system 100 configured to signal the detection of electrocardiogram signal features potentially associated with heart conduction system injury, according to some embodiments of the present disclosure.

In some embodiments, alarm system 100, in its operating configuration, comprises:
Body surface electrodes 105;
ECG circuitry 110, configured to receive the signals from the body surface electrodes 105 and convert them to recorded data of a body surface ECG;
Processor 120, configured to analyze the recorded body surface ECG for changes potentially indicative of cardiac block; and
Indicator device 130, configured to produce an alarm indication according to the analysis produced by processor 120.

In some embodiments, the body surface electrodes 105 comprise at least two or at least three limb-positioned electrodes. In some embodiments, the body surface electrodes 105 comprise one or more (e.g., up to six) precordially-positioned electrodes (that is, positioned on the anterior chest wall).

In some embodiments, ECG circuitry 110 is configured to sampling the analog signal of the body surface ECG at a sufficiently small intervals of time to allow precise detection of selected features of the ECG signal that, upon a change in their relative timing, trigger an alarm from indicator device 130. The sampling frequency of the body surface ECG is, for example, at least 1 kHz. Optionally, the sampling frequency is at least 100 Hz.

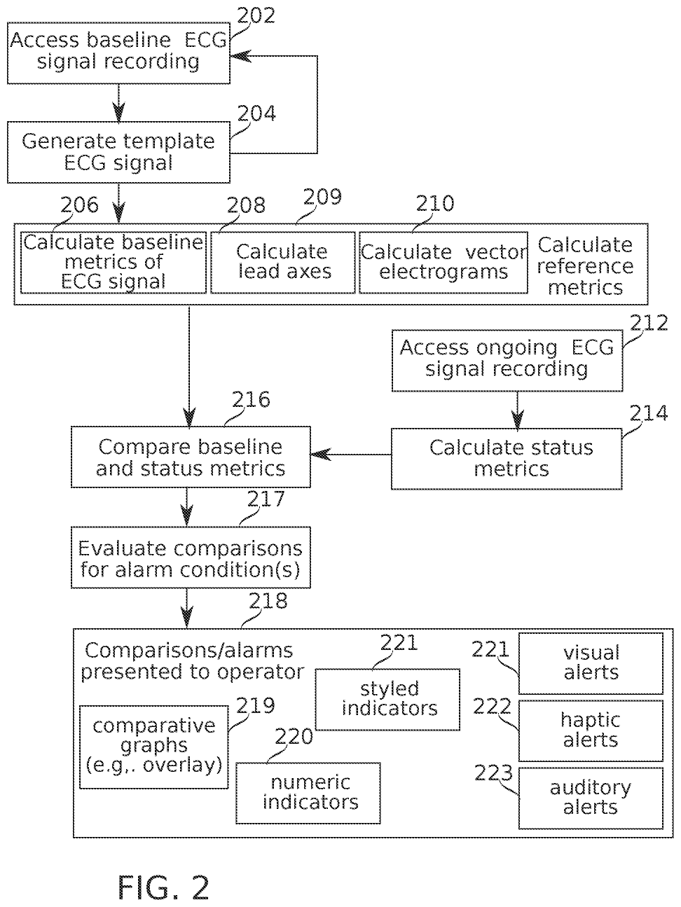

In some embodiments, processor 120 is under the control of digital memory-stored instructions which, when executed, cause an impulse transmission change detection algorithm to be carried out, for example, as described in relation to FIG. 2.

Reference is now made to FIG. 1B, which is a block diagram schematically representing components of an alarm system 100, configured to signal the detection of electrocardiogram signal changes potentially associated with heart conduction system injury, according to some embodiments of the present disclosure. System 100 of FIG. 1B is an example of a configuration of a system 100 in more of its particulars as also described in relation to FIG. 1A.

Operations described herein in relation to the operations of one or more hardware processor(s) 120 of a computing device 402C are performed by hardware processor 120 executing code instructions 406A stored in a memory 406C.

Computing device 402C may be implemented as, for example, a client terminal, a server, a computing cloud, a virtual server, a virtual machine, a radiology workstation, a workstation installed within a catheterization laboratory, a mobile device, a desktop computer, a thin client, a smartphone, a tablet computer, a laptop computer, a wearable computer, glasses computer, and/or a watch computer.

Multiple architectures of system 100 based on computing device 402C may be implemented. For example, computing device 402C may be implemented as an existing device (e.g., client terminal) having software (e.g., code 406A) that performs one or more of the operations described, for example, in relation to FIGS. 2, 6, 8, and/or 9. For example, code 406A is installed, in some embodiments, on a computer conventionally existing in a catheterization/interventional lab. In another implementation, computing device 402C may be implemented as a dedicated device, having software (e.g., code 406A) installed thereon.

In some embodiments, computing device 402C storing code 406A is implemented as one or more servers; for example, a network server, a web server, a computing cloud, a virtual server, a radiology server, and an interventional laboratory server. The implemented one or more servers provide services based on one or more of the operations described, for example, in relation to FIGS. 2, 6, 8, and/or 9 to one or more client terminals 421 over network 420. Client terminal 421 in some embodiments, comprises a terminal located remotely from computing device 402C, for example, an interventional/catheterization laboratory client having access to computing device 402C acting as a server. In such an implementation, for example, remotely generated alarms and/or data indicative of measured indications of sub-threshold heart block are transmitted from respective client terminals 421 to computing device 402C over network 420 for distribution to one or more additional client terminals 421. Data are optionally transmitted from computing device 402C over network 420 to one or more client terminals 421 for presentation on a display associated with the respective client terminal 421.

Hardware processor(s) 120 (also referred to herein simply as “processor(s)” may be programmed to execute code 406A for implementing the operations described, for example, in relation to FIGS. 2, 6, 8, and/or 9.

In some embodiments, hardware processor(s) 120 may be implemented as a central processing unit(s) (CPU), a graphics processing unit(s) (GPU), field programmable gate array(s) (FPGA), digital signal processor(s) (DSP), and/or application specific integrated circuit(s) (ASIC). Processor(s) 120 may include one or more processors, which may be homogenous or heterogeneous, which may be arranged for parallel processing, as clusters and/or as one or more multi core processors.

Memory 406C stores code instructions (e.g., in digital form) executable by processor(s) 120. Memory 406C may be for example, a random access memory (RAM), read-only memory (ROM), and/or a storage device, for example, non-volatile memory, magnetic media, semiconductor memory devices, hard drive, removable storage, and optical media (e.g., DVD, CD-ROM). Memory 406C stores code 406A.

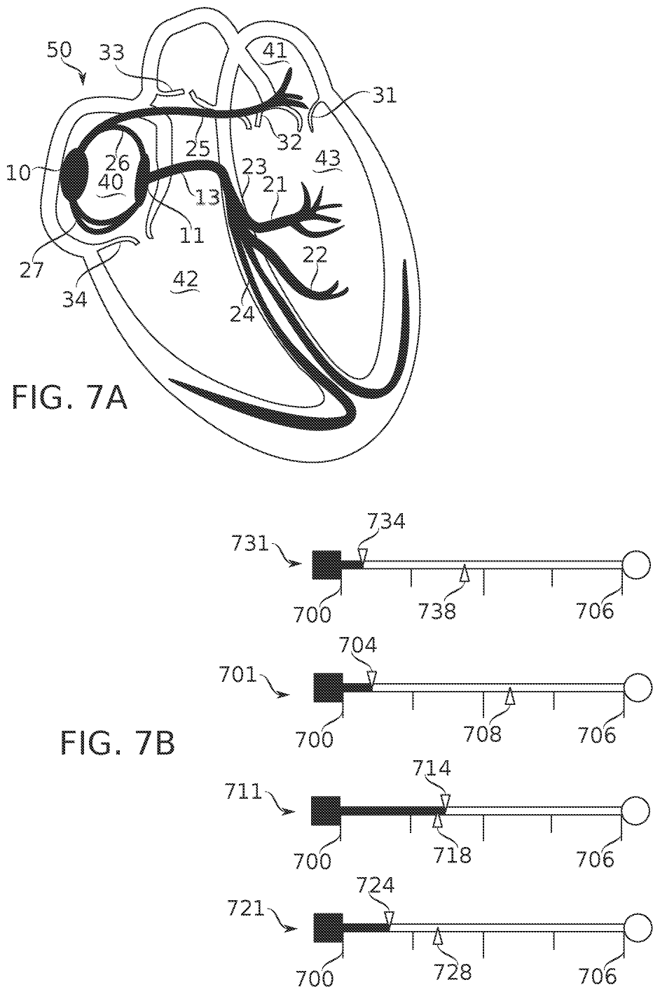

Computing device 402C may include an output interface 430 for communicating with one or more indicator devices 130. The indicator device optionally comprises display 432, for example, a screen or a touch screen. Optionally, alerts and/or sub-threshold heart condition block monitoring information (e.g., a level of sub-threshold heart conduction block, optionally associated with one or more particular heart conduction system structures) is presented on display 432. Optionally, the information is displayed as a heart model or indicator (e.g., as described in relation to FIGS. 7A-7B). Additionally or alternatively, output interface 430 comprises one or more sound, vibration, and/or light producing devices, to produce, e.g., visual, haptic, and/or auditory alerts 221, 222, 223 (FIG. 2).

Optionally, computing device 402C includes a network interface 418, for communicating with server(s) 422 over a network 420, for example, to obtain code 406A such as an updated version of software, and/or transmit alarm and/or ECG data to server(s) 422. The data may be, for example, indicative of sub-threshold heart conduction block. Network interface 418 may be implemented as, for example, one or more of, a network interface card, a wireless interface to connect to a wireless network, a physical interface for connecting to a cable for network connectivity, a virtual interface implemented in software, network communication software providing higher layers of network connectivity, and/or other implementations.

Network 420 may be implemented as, for example, the internet, a local area network, a virtual network, a wireless network, a cellular network, a local bus, a point to point link (e.g., wired), and/or combinations of the aforementioned.

Optionally, a user interface 424 is in communication with computing device 402C. User interface 424 may include a mechanism for the user to enter data, for example, a touch screen, a mouse, a keyboard, and/or a microphone with voice recognition software. In some embodiments, the user may enter data via a graphical user interface (GUI) presented on display 432, where the GUI acts as user interface 424.

It is noted that one or more interfaces 418, 426, 430 may be implemented, for example, as a physical interface for example, cable interface, wireless interface, network interface, and/or as a virtual interface for example API, SDK. The interfaces may each be implemented separately, or multiple (e.g., a group or all) interfaces may be implemented as a single interface. In some embodiments, ECG interface 426 comprises electrode attachment ports for at least 10 body surface ECG electrodes. In some embodiments, ECG interface 426 has electrode attachment ports for no more than 6 body surface ECG electrodes, for example, 6, 5, 4, or 3 body surface ECG electrodes. This is a potential advantage for use with a relative simplified ECG recording setup which may be more familiar to medical teams specialized for treating, e.g., structural heart disease; there may also be a potential advantage in reduced cost and/or increased local availability of ECG recording equipment for 3-6 electrode configuration, compared to 10 or more electrode configuration. It's also a potential advantage for simplifying the overall procedure.

Processor 120 may be coupled to one or more of memory 406C, data storage device 408C, and interfaces 418, 426, 430.

Optionally, computing device 402C includes data storage device 408, for example, for storing ECG data. Data storage device 408 may be implemented as, for example, a memory, a local hard-drive, a removable storage device, an optical disk, a storage device, and/or as a remote server and/or computing cloud (e.g., accessed using a network connection).

It is noted that computing device 402C may include one or more of the following components: processor(s) 120, memory 406C, data storage device 408, and interfaces 418, 412, 426, 420, 434, 442, for example, as a stand-alone computer, as a hardware card (or chip) implemented within a current computer, for example, catheterization laboratory computer, and/or as a computer program product loaded within the current computer.

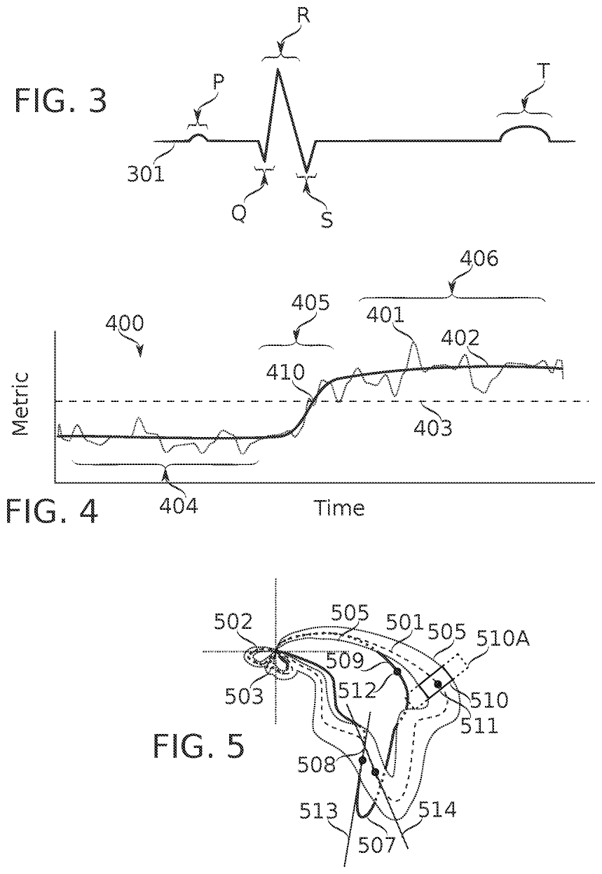

Reference is now made to FIG. 2, which schematically represents an algorithm configured for detection of changes in the relative timing of certain events present within a recorded body surface ECG signal, according to some embodiments of the present disclosure. Reference is also made to FIG. 3, which schematically represents features of a single body surface ECG cycle, according to some embodiments of the present disclosure.

At block 202, in some embodiments, the processor accesses a baseline ECG signal recording (which may be a baseline portion of an ongoing ECG signal recording). The baseline recording may represent ECG signals recorded over some previous period; for example, over the course of about 10 seconds or about 100 seconds.

At block 204, in some embodiments, the baseline signal is converted to a template ECG signal. In some embodiments, the conversion comprises combining (e.g., summing or averaging) individual cycles of the baseline ECG signal recording. The combining may follow one or more procedures which asset in registering ECG signals from individual heartbeats to each other so that they match in phase in at least one of their portions.

In some embodiments, for example, a template window is defined, and adjusted to include a duration of, for example, about 90% of the average interval between two ECG signal heartbeat cycle landmarks. In some embodiments, the average interval between the peak of two R waves (RR interval) is used as a registration landmark. If the baseline signal recording had an average RR interval of 1000 ms, for example, the template window is optionally set to be 900 ms.

In some embodiments, the discarded 10% of the cycles (or other discarded cycle portion) is selected to occur at times which are relatively uninformative as to the velocity of conduction occurring e.g., between and/or including the times of the P wave and the QRS complex. In some embodiments, for example the template window centers on the R wave peak of each heartbeat cycle, and extends for about 45% of the heartbeat cycle before and after the R wave peak. It should be understood that the template window does not necessarily place the R wave peak in its the center, and that the R wave peak is not necessarily the landmark feature used to align signal recordings. Using a landmark feature (optionally more than one, as next described) has the potential advantage of aligning individual heartbeats of the baseline at or near one or more heartbeat cycle events important to detecting conduction changes; potentially minimizing “blurring” due to averaging.

In some embodiments, the combining is performed after time-base adjustments to account for ongoing variations in heart rate. The time-base adjustment may comprise, for example, stretching or shrinking (in time) individual cycle ECG signals to time-align key features; e.g., features selected from those shown in FIG. 3. Additionally or alternatively, ECG signals from individual heartbeats are converted from the elapsed-time domain (units, e.g., of seconds) to a domain of heartbeat cycle phase (units, e.g., of percent or phase angle).

There may be a normal non-linear relationship between heart rate and the phase intervals of certain ECG signal events such as those marked in FIG. 3. For example, of two unequal heartbeat cycles, a 20% longer heartbeat cycle may occur with a smaller than 20% extension in the interval between the P wave and the QRS complex. In some embodiments, the template ECG signal also accounts for those non-linearities, e.g., by averaging individual heartbeats from the baseline signal in a plurality of separate bins according to their elapsed-time duration.

Later comparisons for the sake of detecting changes are made, in some embodiments, with respect to the bin which “most resembles” (e.g., has the most similar elapsed-time duration for its constituent heartbeat cycles) the ECG signal being analyzed for changes. Additionally or alternatively, interpolation functions are generated to estimate how the normal non-linearities change from bin to bin; later comparisons may be based on the interpolation function.

It may be noted that during a surgical procedure, heart rate is generally anticipated to stay within a small enough range (at least over intervals of second to a minute or so) that these non-linearities can be treated as insignificant for purposes described herein. However intraoperative heart rate variations may nevertheless occur, and sensitivity and/or accuracy of detection may improve when normal non-linearities are accounted for.

It should be noted that the template ECG signal optionally comprises a plurality of time series recordings, corresponding to different ECG leads. “Leads” may be understood as data recordings, distinguished from the “electrodes” used to sample the data; e.g., a standard 12-lead ECG comprises leads derived from measurements made using ten placed electrodes. For recording some leads, electrodes are used in pairs (one exploring electrode and one reference electrode). For other leads, electrodes are used in other combinations; for example, one exploring electrode and two or three reference electrodes. In a standard 12-lead ECG, two-electrode leads extend between pairs of the so-called “limb” electrodes, and comprise the Lead I, Lead II, and Lead III leads. Additional leads use the limb electrodes in combinations of three, with two electrodes combining as the reference, and one as exploratory; these are known as the AV, AV, and AVF leads. Remaining leads use all three limb leads as the reference, with the exploratory electrode are placed on the chest nearer the heart; the six leads here are known as V1, V2, V2, V4, V5, and V6. Any number of leads can be used, for example, single lead ECG, 3-lead ECG, 6-lead ECG, 7-lead ECG, 8-lead ECG, 12-lead ECG, or a greater number of leads. There is no particular limitation to leads using standard electrode placements or leads, although these will generally be preferred for the sake of reproducibility, standardization, and/or familiarity.

The different leads (recordings) are expected to have different features, e.g., different amplitudes and/or timings of signal features. Beat-to-beat temporal alignment adjustments, insofar as they are performed, are generally applied consistently to all the signals recorded together during an individual heartbeat cycle, since they all relate to the same events.

The recording epoch used to generate the template ECG signal comprise, for example, a fixed selection of heartbeats (optionally updated periodically), a cumulative selection of heartbeats, or a rolling selection of heartbeats (old heartbeats discarded as new beats are recorded). Optionally, contributions of heartbeats are weighted according to recency. The cyclic arrow returning from block 204 to block 202 represents optional dynamic updating of the baseline ECG signal recording data used to generate the template ECG signal.

The interval between the current time (of ongoing ECG signal recording, described in relation to block 212) and the end of the recording epoch of the template ECG signal is optionally selected to be any value from a single heartbeat back to the beginning of the procedure. This interval is chosen, in some embodiments, to allow relatively sudden prolongations (e.g., those developing within about 10 seconds) to be easily distinguished from slower variations which may be occurring as part of normal background changes in heart activity.

Optionally, multiple template ECG signal epochs are generated and maintained; for example, a more recent epoch (e.g., completed within the last 10-60 seconds, and/or immediately before a triggering event) to emphasize fast changes, and an older epoch (e.g., completed two or more minutes previously) to emphasize slower changes. In particular: in some embodiments, the template ECG signal is established using data recorded at the beginning of a procedure, and remains the template throughout the procedure. Additionally or alternatively, in some embodiments, a template ECG signal is updated at selected times; for example, updated upon a triggering signal that a risky surgical action has begun, using baseline ECG signal recording data from the 10, 100 or other number of heartbeat cycles recorded prior to the triggering signal being issued. Additionally or alternatively, in some embodiments, a template ECG signal is regenerated on a rolling average basis, discarding and/or reducing the weight of older data, and incorporating newer data as it becomes available.

At block 209, in some embodiments, reference metrics are calculated from the template ECG signal of block 204. Within block 209, the operations of blocks 206, 208, and 210 provide non-exhaustive examples of different optional methods of calculating metrics from the template ECG signal which can be used to evaluate changes in the ECG features which are potentially a result of insult or injury to the heart's electrical system, as may occur inadvertently during treatments of structural heart disease.

Optionally, at block 206, in some embodiments, the template ECG signal is analyzed to calculate baseline metrics.

Signal features indicated in FIG. 3 include:
The P wave, associated with atrial depolarization and subsequent contraction.
The QRS complex, comprising the upward signal deflection R between downward deflections Q and S. It is associated with the events of ventricular depolarization and contraction.
The T wave, associated with ventricular repolarization.

The signal features schematically represented in FIG. 3 can be used to calculate a variety of metrics characterizing temporal features of the single-beat template ECG signal timecourse, including, for example, the onset (e.g., time of rise to at least 10% above baseline 301), offset (e.g., time of return to less than 10% above baseline 301), and/or peak times of any one or more of the P, R, and T waves. Other landmarks include, for example, the times of the onset peak and/or offset of the negative-going Q and S waves, although since these waves blend with the positive-going R wave, some offset/onset times may be considered to coincide. Additionally or alternatively, temporal landmarks are generated based on first or subsequent derivatives of the ECG signal, e.g., the relative times of maximum change in P, Q, R, S, and/or T waves.

Differences between any of the relative times can be calculated to arrive at baseline intervals characteristic of various ECG signal features; for example:
P wave duration (between P wave onset and P wave offset).
R wave duration (between R wave onset and R wave offset).
T wave duration (between T wave onset and T wave offset).
PR interval (between the onset of the P wave and the onset of the Q wave, (or R wave in the absence of a Q wave) while noting that any interval between a P wave feature and a QRS complex feature such as the R wave peak is optionally used additionally or alternatively).
QT interval (between the onset of the Q wave and the offset of the T wave, while noting that any interval between a T wave feature and a QRS complex feature such as the R wave peak is optionally used additionally or alternatively).

The intervals are optionally calculated separately for the different leads of the ECG.

Optionally, at block 208, one or more lead ECG axes are calculated; for example, the limb lead axis and/or the precordial lead axis. Typical conventions used in ECG axis calculation operate to estimate vectors “pointing” within their vector space in directions corresponding with the actual spatial arrangement of the leads used in the ECG recording, and/or to “virtual” leads that combine measurements for actual leads.

Among the methods of calculating lead axis, a qualitative approach evaluates for a certain lead the comparative amplitudes of the R wave (above baseline 301) and the S wave (below baseline 301). By convention, the “axis” points generally (within)±90° toward the exploring electrode if the R wave amplitude is larger (positive), generally away from the exploring electrode if the S wave amplitude is larger (negative), and approximately orthogonal to an axis extending from the heart to the exploring electrode if the S and R waves are of the same amplitude (isoelectric or equiphasic). Combining lead axis evaluations of two or more leads allows refining these types of estimations to the shared quadrant region consistent with each lead axis evaluation alone. Optionally, individual lead axis evaluation is quantified (e.g., according to the magnitude of differences measured, and not just the sign), potentially allowing finer calculation of the combined vector direction. Similarly, vector magnitude is optionally calculated according to R and S wave amplitudes.

Cross-correlations of vectors can be used to determine differences and/or similarities, e.g., of a reference vector calculated for a baseline period, and a test vector calculated for a period of ongoing ECG signal recording.

There is no particular limitation to comparison of the S wave and R wave. Axes can be calculated with respect any time-window region, for example a window including the P wave and/or T-wave.

This type of analysis may be applied as an adjunct or alternative to the interval analyses of block 206.

Optionally, at block 210, vector electrograms are calculated, e.g., the limb lead vector electrogram, the precordial lead vector electrogram, and/or the composite lead vector electrogram. In contrast with the ECG axis calculation, vector electrocardiograms represent the magnitude and orientation of the heart's electrical activity as they change throughout a cardiac cycle. The vector electrogram can receive information contributions from several leads, and since each of those leads itself has an axis in space, the components of the vector electrogram can also be defined in three dimensions.

The evolution of a vector electrogram through the period of a heartbeat can be graphed using the various lead recordings to generate a 3-D plot, and/or a 2-D plot which projects the 3-D plot to a plane. The plot shows “vector loops” traced out by the tip of the electrical vector as its angle and magnitude changes throughout time. The vector loops correspond to the periods including the excursions from baseline that occur during the heartbeat cycle, corresponding, for example, to the P wave, QRS complex, and T wave.

At block 212, in some embodiments, ECG signal data is accessed which indicates ongoing (e.g., present and/or recent) electrical activity of the heart. This ECG signal data may be accessed as single-beat data, and/or in a combined form (e.g., as signal data combined, for example, as described for the template ECG signal data with respect to block 204). Longer combination epochs potentially slow the detection of sudden events, but may have the advantage of averaging out the “noise” of irrelevant changes. Optionally, both single-heartbeat and multiple-heartbeat new ECG signal data is used. Optionally, multiple overlapping epochs of new ECG signal data are tracked at once.

At block 214, in some embodiments, any or all of the metrics of blocks 206, 208, 210 (or another metric calculated as a reference in block 209) are calculated for the new ECG signal data accessed at block 212.

At block 216, in some embodiments, the new ECG signal data metrics are compared to the template ECG signal metrics.

At block 217, in some embodiments, the comparison results are themselves evaluated respect to one or more alarm conditions that may be triggered by comparison results wherein the new ECG signal metrics diverge from the template ECG signal metrics. Alarm conditions are further discussed next in connection with block 218.

At block 218, in some embodiments, indications are presented to an operator (e.g., an operating physician) based on detected feature changes which, according to comparison results, indicate prolongation of intervals associated with signal propagation. The indications are selected to emphasize, for the operator, changes potentially indicative of heart conduction block.

Examples include comparative graphs 219, for example:
One or more images comprising graphs representing the template ECG signal, e.g., as a lead trace, lead axis vector, and/or vector electrogram.
One or more images comprising graphs representing the new ECG signal, e.g., as a lead trace, lead axis vector, and/or vector electrogram. Optionally, corresponding images of the template ECG signal and the new ECG signal are superimposed, allowing differences to be readily detected.
When there are multiple epochs recorded (whether from older and/or newer ECG signal data), there may be a multiplicity of superimposed images shown simultaneously. When no change is happening, the images may tend to align and “hide” one another, within the limits of the normal variability and the normalization procedures being used. As changes develop over the course of several heartbeats, there may be a corresponding increase, e.g., in the apparent thickness and/or number of lines being shown, as positions diverge.

Any or all of the images may be updated continuously, for, example updated at each heartbeat.

In some embodiments, numeric indications of metrics and/or metric comparison results 220 are shown. In some embodiments, one or more event intervals may be identified and numerically or otherwise labeled so as to facilitate a time-based comparison of the template ECG signal and the new ECG signal. Compared values may be shown, for example, absolutely and/or or relatively (e.g., as a percentage change). Styling e.g., by use of color, size, and/or another visual indication may be used to emphasize changes such as the degree of prolongation. The styling may be continuously changing as a function of the comparison result, and/or changing abruptly upon passing a threshold of, e.g., prolongation. A time course of numeric values such as event intervals may be shown as a graph, e.g., a graph showing interval values since the beginning of the procedure.

Vector-based displays of ECG signal data may be presented to use time as a parametric variable, wherein time changes as function of distance along the trace of a function without corresponding to a spatial axis for the graph.

The loops of a vector electrogram, for example, emphasize a “shape” which indicates ECG recording data in terms of magnitudes and angles, rather than in terms of intervals. Propagation delays may cause different heart regions to, e.g., depolarize at slightly different relative times, diverting the path of the vector electrogram into new directions and/or changing its magnitude. As also described for indications of changing event intervals, metrics of loop changes are optionally indicated numerically; for example as numbers estimating loop-to-loop distances in vector space, and/or as comparisons of perimeter lengths, areas, and or volumes “swept” by vector loops.

Examples of this are also discussed in relation to FIG. 5.

Additionally or alternatively to image- and/or measurement-type indications, one or more alerts (e.g., visual, haptic, and/or auditory alerts 221, 222, 223) may be presented to a user when certain conditions are met. Users may be able to modify options for configuring the alerts, e.g., a threshold of prolongation before the alert is activated. Optionally, for example, the alerts are threshold driven; for example, a threshold of a 10% prolongation in an interval or larger. An operator, in some embodiments, is able to set a threshold of prolongation beyond which an alert is activated. Such a configuration is described, for example, in relation to FIG. 4. Thresholds may be jointly defined by a plurality of factors, for example as represented by entries in a table or other multi-factorial data structure.

In some embodiments, alerts are configured to become more noticeable as a function of a greater duration that a threshold is exceeded, and/or as a threshold is exceeded by larger amounts, e.g., prolongation more than 15%, 20%, or 40%. In some embodiments, the threshold includes a rate of change, parameter; e.g., an average rate of change of 0.5% or more per second over at least 10 seconds, an instantaneous rate of change exceeding some threshold and/or number of standard deviations (e.g., three standard deviations) of changes seen until now in the procedure.

Where, in the preceding, an example of “prolongation” is given for a changing variable, it should be understood that changes in another metric may be substituted; for example, a change in the direction of an axis direction (e.g., of the P wave, R wave, T wave, or QRS complex), or a change in a metric characterizing the morphology of a loop of a vector electrogram.

Alarms can be implemented to stimulate any suitable sensory modality: audible (e.g., tones, verbal warnings), visible (e.g., image display or indicator lamp-based), and/or haptic (vibrations of a tool, surface, and/or user-worn apparatus).

### Threshold-Sensing Alarm System

Reference is now made to FIG. 4, which schematically illustrates elements of a threshold-style alarm system, according to some embodiments of the present disclosure.

Graph 400 shows the graph of an ECG metric (calculated, for example, as described for any of the metrics discussed in relation to block 209 of FIG. 2) as a function of time. Trace 401 represents raw moment-to-moment (e.g., heartbeat-to-heartbeat) values of the metric, while trace 402 represents filtered (e.g., time-averaged) values of the metric.

In the example, baseline 404 corresponds to all or a portion of the time of baseline ECG signal recording, for example as described in relation to blocks 202 and 204. Within region 405, the metric's value is changing; in this case, rising, for example as may occur when an interval metric suffers prolongation due to insult or injury of the heart's conduction system. Region 406 shows a period of continued elevation of the metric.

This is the sort of graph which may be shown to an operator (a heart surgeon, for example) directly, allowing the operator to see visually that a certain metric has undergone a change. The operator may be able to correlate this visual indication with their own actions to understand that a potential complication related to heart block is arising and/or has arisen during the procedure.

In some embodiments, a threshold level 403 may be set so that an additional alert and/or alarm can be raised at the threshold crossing point 410. In some embodiments, the threshold is set automatically, e.g., based on noise (variation) levels present in the metric as determined for the period of baseline recording—for example, above three standard deviations of the noise of the metric during the baseline recording. Thresholds are optionally set to apply to one or both of the filtered and raw metric outputs. Using the raw metric provides a potential advantage for rapid detection of changes, potentially at the expense of responsiveness. A filter metric may indicate problem less quickly, but with a lowered sensitivity to normal fluctuations in the value which could result in a false alarm.

Vector Electrograms and Metrics Derived from Them

Reference is now made to FIG. 5, which schematically illustrates a graph (in two dimensions) of baseline and potentially alarm-triggering vector electrograms, according to some embodiments of the present disclosure.

The three loops 502, 501, 503 of FIG. 5 correspond respectively to the P, QRS, and T waves of the template ECG signal. Motion once along each of these three loops in sequence moves also in time through electrical vectors corresponding to a single heartbeat cycle, each vector ending on the loop, and beginning at the origin. For moments when the signal amplitude is zero, the vector electrogram stays near the origin. As signal amplitude rises, the loops extend further and further from the origin, in a direction corresponding to the directionality of the electrical signal at that moment. A loop's shape overall is indirectly characteristic of the pattern of the waves of electrical depolarization and repolarization that spread over the heart as it beats. Accordingly, a disruption to the timing of signal propagation in the heart itself becomes a disruption that may appear as movement away from the baseline shape of the vector electrogram.

FIG. 5 shows an example of how this distortion may be presented and/or measured. Loops 505 represent an “alert range” that has been defined for the vector electrograms 505.

In this case, the alert range at any given moment is defined as extending orthogonally to the direction of movement of the vector, to a distance (in vector space) which is increasing (e.g., proportionally) as the magnitude (distance from the origin) also increases. This increase as a function of magnitude is optional, but has the useful property of emphasizing relative changes, which allows smaller absolute changes in the smaller loops (e.g., the P and T wave loops 502, 503) to have detectability more comparable to fractionally similar absolute changes in magnitude in the QRS loop 501. In two dimensions, the momentary alert range is a line segment, and the two loops 505 represent the positions of the ends of that line segment as it sweeps along, e.g., loop 501—the alert envelope. In three dimensions, the momentary alert range would be, e.g., a disk that sweeps out an alert envelope comprising a distorted tubular-type volume.

The abnormal vector electrogram 507 comprising regions 508 and 509 is partially within (e.g., heavy dotted-line region 508) and partially outside (e.g. heavy solid-line region 509) the alert range envelope. The alarm is optionally set so that any excursion from the alert envelope leads to an alarm being raised. Additionally or alternatively, there may be alarm levels set based on the severity of the excursion—e.g., for what fraction of the loop and/or to what time-integrated distance the excursion occurs. Evaluation of the severity of an excursion may additionally or alternatively be based on the first or higher-order derivative of the vector electrogram. For example, even though the lower tip of region 508 (intersecting loop 501) is inside the magnitude and direction-based alert envelope, the vector electrogram in these location is still moving in the wrong direction (crossing instead of along loop 501). Slope indications 513 (on the abnormal lop of vector electrogram 507) and 515 (on the baseline vector electrogram) indicate corresponding points in time that are nonetheless distinguishable by the direction of vector movement.

Additionally or alternatively, the alert envelope is defined with respect to parametric time (heartbeat phase). For example, the alert envelope portion associated with a given single vector of the baseline vector electrogram may also be defined to allow a certain amount of difference forward and backward in time (phase), defining a roughly, e.g., rectangular (in 2 dimensions) or cylindrical (in 3 dimensions) alert envelope. For example, points 512 (on an abnormal loop of vector electrogram 507) and 511 (on baseline QRS loop 501) in the drawing correspond in time. But not only is point 512 outside of the static alert region, it is more particularly lagging the temporal “box” 510 defined behind (and optionally also in front of) point 511. Even if the vector direction- and magnitude-based alert envelope of 510 were widened arbitrarily (e.g., to box 510A), it would still not include point 512.

These considerations illustrate how a vector electrogram may be converted into a “change detector” which is potentially sensitive to changes in several vector electrogram features at once; e.g., magnitude, angle, slope, and parametric phase (time).

Although graphical representations of these differences may be difficult to interpret by eye, they are reach readily converted to numerical metrics allowing use of a visually simpler scheme, e.g., the timecourse-thresholding method described in relation to FIG. 4. For example, any of magnitude difference, angle difference, slope difference, and parametric phase lag, can be used as scalar values that provide the “metric” axis of FIG. 4. Optionally, multiple scalar metrics are combined into new single scalar metric, e.g., by combining them to a new vector (with components of, e.g., magnitude, angle, slope, and phase) and finding the magnitude of that new vector.

### Patient-Personalized Warning Configuration

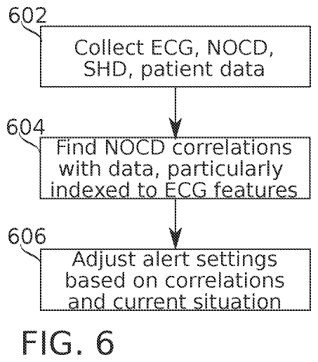

Reference is now made to FIG. 6, which schematically outlines a method of setting alarm levels for the device of FIGS. 1A and/or 1B, according to some embodiments of the present disclosure. In some embodiments, the method has outputs which are additional or alternative to the setting of alarm levels, for example, warning a physician in advance of a likelihood of an NOCD complication, enabling pro-active measures to be taken.

The likelihood of NOCD induction during an SHD is potentially dependent on multiple factors, for example:
baseline condition of heart conduction system,
patient age,
patient sex,
patient habitus,
any parameter of the SHD procedure itself, e.g., specific techniques used,
device used,
ancillary devices used (for example, sheath, wire, or balloon),
concomitant diseases (for example diabetes),
the operator performing the procedure,
center performing the procedure, and/or
level of real time monitoring during the procedure.

In some embodiments of the present disclosure, default and/or recommended threshold levels for alerts and/or alarms are set based on prior experience findings correlated with one or more of these, or another factor.

At block 602, in some embodiments, data is collected from procedures performed a clinical study setting, including procedure outcome, patient demographic data, features of the baseline ECG and/or ongoing ECG signal recordings (and/or the recordings themselves), and/or intervention information (e.g. implant type, and/or size of the implant).

At block 604, in some embodiments, data mining tools are applied to determine possible correlations between the information collected in block 602, and observed development of heart conduction system injury. In some embodiments, the tools used include support vector machines (SVM) and/or other tools which develop a predictor that can be updated momentarily according to the momentary ECG information.

At block 606, in some embodiments, alert range(s) are set based on the correlations determined in block 604. In some embodiments, the goals of selection comprise (1) setting the threshold low enough that no NOCDs are missed, while (2) avoiding false alarms which may result in a physician learning to disregard warnings as a matter of course. Since there may, in some cases, be no setting which perfectly satisfies both goals, the selection may weight the two goals against each other (e.g., balancing allowing a small percentage of NOCDs missed against a large potential reduction in false alarms). In some embodiments, the physician is allowed to choose the tradeoff, e.g., by adjusting the sizes of one or more alert ranges while being informed of the predicted result on false positives and false negatives.

Apart from alerts, there may also be presented one or more indications of the likelihood of developing conduction system injury based on the predictor.

It is noted that FIG. 2 relates to the determination of differences from a template ECG signal. Additionally or alternatively, in some embodiments, alert ranges are set according to the current data-described situation (that is, data including the ongoing ECG signal as well as key predictor factors) being similar to situations which, according to the data and analysis of blocks 602-604, are associated with NOCD. This has the potential advantage of encouraging the physician to take extra precautions in high-risk cases, e.g., proactively pre-implanting a pacemaker to the heart of a patient at special risk for developing NOCD during the treatment. Thus, the setting of alert ranges in block 606 is optional.

### Anatomy of the Heart Conduction System

Reference is now made to FIG. 7A, which schematically illustrates anatomical elements of the conduction system of a heart 50. As reference, heart chambers including the right atrium 40, left atrium 41, right ventricle 42 and left ventricle 43 are shown, along with the tricuspid valve 34, pulmonary valve 33, mitral valve 31, and coronary valve 32.

In a normally functioning heart, electrical impulses initiate at sinus node 10. An impulse pathway leading to the ventricles travels down internodal pathways 26, 27 to the AV node (atrial-ventricular node) 11. Another impulse pathway leads to the left atrium along Bachmann's bundle 25.

From the AV node 11, impulses continue along the bundle of His 13, and soon branch between the right bundle branch 24 and the left bundle branch 23. The left bundle branch 23 ramifies further into the anterior fascicle 21, the posterior fascicle 22, with each of the branches and bundles themselves eventually ramifying into numerous Purkinje fibers spread throughout the myocardium.

Given their structural order, it may be understood that blocks earlier along the conduction pathway may affect transmission to a plurality of downstream structures, and the heart chambers or portions thereof that they stimulate. For example, damage to the bundle of His 13 can affect electrical conduction to both ventricular chambers 42, 43; damage to the left or right bundle branches 23, 24 may selectively affect the left or the right ventricle 43, 42, respectively. Moreover, the location of damage along the left bundle branch 23 can determine whether transmission through one or both of the anterior fascicle 21 and posterior fascicle 22 is also affected. Anterior fascicle 21 and posterior fascicle 22 can also each become impaired independently of other conduction pathways dependent on the left bundle branch 23.

Accordingly, ECG timing delays consequent to full or partial conduction block can be interpreted, according to their pattern, in terms of particular regions which are the likely locus of injury.

### Heart Block Alarm Indicator and Control Example

Reference is now made to FIG. 7B, which schematically illustrates user interface elements for indicating existing and/or incipient heart block, and adjusting heart block alarm settings, according to some embodiments of the present disclosure.

FIG. 7B illustrates a user interface control/indicator for use in some embodiments of the present disclosure.

Each of indicator bars 701, 711, 721, 731 corresponds to a different node, bundle, fascicle, and/or plurality of such structures of the heart conduction system. Examples of the structures include the AV node 11, the bundle of His 13, and the right, left anterior, and left posterior fascicles 24, 21, 22.

For each indicator bar 701, 711, 721, 731: tick 700 (left-hand tick) represents a baseline value of a heart block parameter (also referred to herein as a “reference value”), while tick 706 (right-hand tick) represents a value of the heart block parameter which indicates meeting a predetermined condition of heart block (heart block condition value).

In between, marks 704, 714, 724, 734 indicate a current value of the heart block parameter, each positioned between ticks 700 and 706 according to the fractional distance of the current value from the baseline value, compared to the distance of the heart block condition value from the baseline value.

Trigger marks 708, 718, 728, 738 indicate optional alarm thresholds, which may be set in predetermined and/or user controlled locations. The alarm thresholds select a sensitivity of the system to triggering an alarm. Optionally, alarm thresholds are set identically with the heart block condition value (that is, at tick 706), or at a lower value according to default values, values modified from the default in consideration of auxiliary conditions (e.g., stability of measurements), and/or user preference.

In the example shown, mark 734, representing, for example, AV node and/or bundle of His block, is positioned just above the baseline (0%) tick, and below the alarm threshold set by trigger mark 738.

Mark 704, representing, for example, right bundle block, is positioned a little higher than mark 734 but still well below the 25% block mark, and below the threshold set by trigger mark 708.

Mark 714, representing, for example, left anterior fascicle block, is positioned roughly half-way between the 25% and 50% ticks; and moreover beyond the threshold set by trigger mark 718. In some embodiments, this triggers an alarm indication, e.g. one or more of visual, haptic, and/or auditory alerts 221, 222, 223.

Mark 724, representing, for example, left posterior fascicle block, is positioned also below the 25% tick, and also below the threshold set by trigger mark 728.

This pattern of block indications of FIG. 7B is suggestive of a block induced or developing particularly on the anterior fascicle 21 of the left bundle branch 23. Possible partial involvement of the posterior fascicle 22 suggests that damage may be near to the root of the anterior fascicle 21. The slightly elevated values for the AV node/bundle of His 11 (at indicator bar 731) and the right branch bundle 24 (at indicator bar 701) may be due to a general change in patient state (e.g., heart rate slowdown); such a general factor is optionally taken into account by the physician for interpreting the seriousness of the apparent partial block of the left anterior fascicle. In some embodiments, the alarm threshold (e.g., threshold 718) is optionally adjusted, e.g., upward to assist in discounting the effects of global conduction rate slowdowns (e.g., slowdowns shared at a sub-threshold level among a plurality of channels) which are not likely due to localized injury.

In some embodiments, the baseline setting of each indicator bar 701, 711, 721, 731 is set to a value measured from a patient's heart before potentially risky procedure activities such as device implantation begin. Optionally, the baseline setting is set to a value measured intra-operatively, additionally or alternatively, to a value measured pre-operatively. In some embodiments, the baseline setting is based on a canonically accepted “normal” value (e.g., normal for a person from a population matched to the patient in terms of age, sex, average heart rate, general medical condition, and/or other criteria). In such embodiments, it may still be useful to display the patient's own baseline value as a separate indication, e.g., as another marker. A potential advantage of comparing with respect to both the patient's own baseline and to a population-matched baseline is to emphasize for the physician the absolute size of the available working range for the heart block parameter before the heart block criterion is reached. For example, this can help normalize the range of measurement noise from procedure to procedure, which may assist a physician in attributing measured parameter value variability to measurement instability, or to something actually changing within the heart itself.

There may be other adjustment settings. For example, there may be one or more settings to control weighting of recent measurements to obtain displayed current values. A weighted average of a plurality of recent measurements has the potential advantage of reducing spurious alarm triggering due to measurement error. If weighting is selected so that fewer measurements are used (and optionally just the most recent measurement), time responsiveness of the alarm system may be increased. Optionally, both types of weighting function are used, optionally each with its own threshold. For example, the dark bar of each indicator of FIG. 7B may be set to follow a relative recent measurement period (e.g., the last heartbeat or two), while the sliding indicator 704, 714, 724, 734 may indicate an average over a longer measurement period. Alerts may be provided that alert to any one or more of the indications entering an alert zone and/or remaining in an alert zone (e.g., above a threshold) for a certain period of time. Alerts may be configured to increase in intensity and/or another type of insistence (e.g., repetition) as a function of time spent in the alert zone, and/or as a function of how deeply into the alert zone the indicating value progresses.

It should be understood that particulars of the indicator/control design shown are indicative and not limited to the forms explicitly shown and/or described. For example, progress toward full conduction block may be indicated (additionally or alternatively) by a dial indicator, other filled-in shape such as a circle, numeric indicator, color change indication, icon display, size change, and/or motion (e.g., shaking or pulsing). Indications may be provided, additionally or alternatively, to a non-visual sensory modality, e.g., via sound or haptic outputs.

In some embodiments, the role of the sliders in FIG. 7B is fulfilled using a map of the heart (e.g., like that of FIG. 7A), wherein different parts of the conduction system are shown with a difference (e.g., in color, for example a spectrum of colors ranging from green through yellow and orange to red) according to the value setting the positions of marks 704, 714, 724, 734 in FIG. 7B. This has the potential advantage of indicating more directly to the physician where anatomically the block was caused.

### Determination of Root Causes of Condition Indications

As already mentioned, insults to different parts of the heart conduction system may lead to different patterns of heart blocks. The following table indicates examples of different clinically recognized blockage conditions (left column), indexed to anatomical structures (top row) which are physiologically impaired in their function as a result of the clinical situation. Clinical criteria may be designed to produce determinations in terms of either the left column, or the top row. In some cases, the clinical state and the affected structure are effectively synonymous; e.g., right bundle branch block (RBBB) affects the right bundle. In others, the clinical state has downstream effects; e.g., AV block affects left and right structures. As a result, each structure is associated in the table below with a plurality of different conditions.

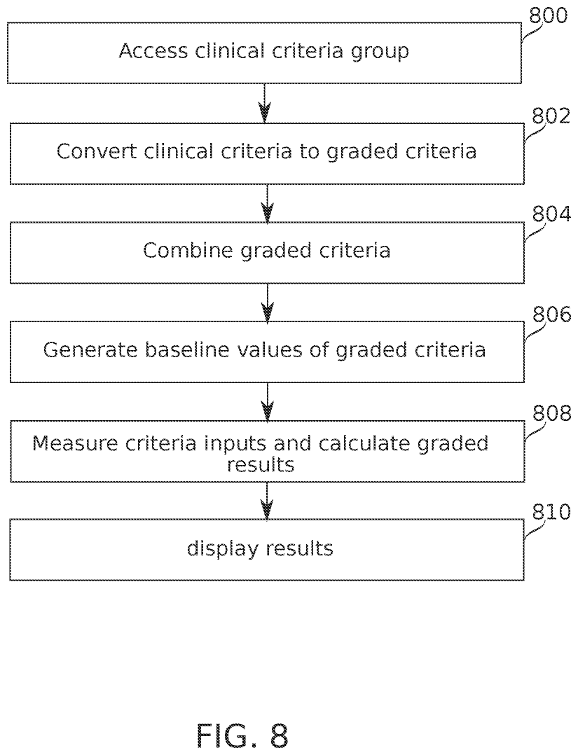

In some embodiments, the clinical criteria groups which are available (e.g., to be converted into graded criteria groups as next described in relation to FIG. 8) are couched in terms of the conditions rather than the structures. The indicator bars of FIG. 7B are each structure associated. Optionally, conversion from condition to structure comprises using the graded condition assessment which gives the maximum value (most impairment), of all the conditions applicable to a given structure.

The abbreviations in the table following are built from a common set of terms: last “B” in each refers to “block”, “R” and “L refer to “right” and “left”, “BB” is “bundle branch”, and “AF” and “PF” refer to anterior and posterior fascicles, respectively.

| | RIGHT | LEFT ANTERIOR | LEFT POSTERIOR |
|---|---|---|---|
| | BUNDLE | FASCICLE | FASCICLE |
| AV BLOCK | Blocked | Blocked | Blocked |
| RBBB | Blocked | | |
| LAFB | | Blocked | |
| LPFB | | | Blocked |
| LBBB | | Blocked | Blocked |
| RBBB + LAFB | Blocked | Blocked | |
| RBBB + LPFB | Blocked | | Blocked |

### Heart Block Criteria

Reference is now made to FIG. 8, which schematically illustrates algorithmic strategies used to convert clinical criteria for block and/or partial block into algorithms for graded assessment of sub-threshold block, and use of those algorithms, according to some embodiments of the present disclosure.

### Calculating and Combining Heart Block Criteria as Graded Indications

The heart block condition value, in some embodiments, is generated in accordance with generally accepted (i.e., data-supported) clinical criteria. There being several distinct types of heart conduction block, a multiplicity of criteria are typically offered to assist in diagnosis, with the criteria being structured as a group so as to lead to a correct diagnosis and avoid incorrect findings: whether spurious altogether; or perhaps close, but still too incorrect or imprecise to guide clinical decision making. Block 800, in some embodiments, represents one or more such structured groups of clinical criteria.

Blocks 802, 804, in some embodiments, represent transformation of the clinical criteria groups of block 800 into an algorithm which produces graded sub-threshold indications of clinical state while preserving main aspects of the logic (and where relevant, comparison values) of the original clinical criteria group. Graded sub-threshold indications are used, in some embodiments, to generate values suitable for use in warning displays and/or alarm triggering, for example as described in relation to FIGS. 1A-B, 2, and 7A-7B.

In some embodiments, the process of transformation is more particularly selected to preserve category results—that is, when a disease state is present, the graded criteria give the same supra-threshold results as the original algorithm, insofar as relevant. However, sub-threshold indications are also provided.

Block 802, in some embodiments, indicates conversion of one or more threshold (true or false) criteria into values having a graded range. This conversion can be performed, for example, by asserting a range existing between whatever the baseline value is and the block threshold value is, and then expressing the criterion as a fractional value between the two ends of the range. For example, the value v of the range-converted criterion may be expressed as

$$v = \frac{{F}_{c} - {F}_{b}}{( T - {F}_{b} )} ,$$

where F_{b} is the baseline value, F_{c} is the currently measured value, and T is the threshold value. Minimum and/or maximum value limits are optionally applied to ensure that the value v remains within the range (0,1).

At block 804, in some embodiments, the graded criteria of block 802 are combined. In some embodiments, where two or more numerical value-based criteria are linked by an AND in the clinical evaluation criteria, just the lowest value is taken (the one least indicative of block).

Optionally, another combining method is used. For example, values may be multiplied together and then a root of order corresponding to the number of values is calculated from the result (e.g., \sqrt{ab} for two values,

$$\sqrt[3]{a b c}$$

for three values).

Where two or more numerical value-based criteria are linked by an OR in the clinical evaluation criteria, optionally just the largest value is taken (the one most indicative of block).

Optionally, a criterion can be inserted as an intensifier; i.e., to a degree that it is present, it can be used to adjust the significance of another criterion or combination of criteria, without becoming itself either a sufficient or required criterion (e.g., the selected minimum or maximum value of an AND or OR combination). For example, if the clinical block-assessment criteria include phrasing like: a ESPECIALLY IF b, a OCCASIONAL LY WITH b or a TYPICALLY WITH b, then optionally the value vₐ=f(a) used as a value for range-converted criterion a is adjusted using an additional range-converted criterion v_{b}=f (b) for criterion b. For example, the intensified form may use a function

$${v}_{a} = \sqrt[1 + kf ( b )]{f ( a )} ,$$

where optionally k≅1, or another value depending on how diagnostic criterion b affects the significance of criterion a. This method maintains the 0-1 normalization of vₐ. If k<0, then criterion b becomes an intensifier with a negative effect, which may be appropriate to use for converting expressions such as a USUAL LY WITHOUT b, a RARE LY TOGETHER WITH b, where the existence of condition b is considered to lower the likelihood of condition a being a significant finding, rather than acting as a positive intensifier. This is optionally used similarly to an AND NOT combination of a plurality of conditions.

Other types of combinations may be used additionally or alternatively. For example, a sigmoid co-function may be used instead of and/or additionally to the just-described root order adjustment function group. In the root-order adjustment form, an additive offset may be used so that kf(b) ranges both above and below zero, allowing it to act as either a positive or a negative intensifier of another criterion according to its own value (e.g., amplitude, distance from threshold, or another metric).

As indicated by lines leading from block 801, Criteria may be rewritten, (before after or while applying the operations of blocks 802 or 804) using rules of Boolean algebra so that they can be combined. It may be noted, for example, that NOT-type criteria encompassing a plurality of sub-criteria can be brought down to the level of each individual sub-criterion using rules of Boolean algebra, i.e.: ¬(aandb)=¬aor¬b and ¬(aorb)=¬aand¬b The “not” conversion (indicated by the symbol ¬) may be implemented, e.g., by flipping a threshold comparison operation from greater-than to less-than. As another example, criteria expressing both upper and lower bounds such as C<F_{c}<B (C and B being threshold values) can be rewritten, e.g., as (C<F_{c})&(B>F_{c}), with each threshold inequality being separately converted to a range expression as described for block 802. Alternatively, the double-bounded criterion can be rewritten in a form such as

$$\vert {F}_{c} - \frac{C + B}{2} \vert < \vert \frac{C - B}{2} \vert .$$

Some criteria may be expressed originally in terms of whether a certain feature is “present” (or not) in one or more ECG leads, without mention of a particular threshold value. Optionally, this is converted to a threshold value using detection parameters which are found to work on example recordings. The parameters may be made adjustable to account for specific recording conditions, e.g., based on amplitude relative to nearby ECG landmarks and the expected heartbeat phase in an ECG lead when the feature would appear. The amplitude may itself be noise-amplitude adjusted, e.g., larger in amplitude if the signal to noise level is lower. Optionally, a template lead is recorded or generated and used to set the standard for “has the feature”. For example, the template lead is used to detect a feature by combining it with recorded data in some data operation: e.g., a subtraction, multiplication, and/or correlation operation. The difference between applying that operation on a “minimum detectable” example of the feature (which optionally corresponds to the template itself), and a baseline lead can then be normalized to generate a corresponding range-converted criterion.

### Examples of Clinical Criteria Used in Heart Block Assessment

Various criteria have been proposed relating to one or more of the heart conduction nodes and/or main conduction fascicles/bundles (the terms “fascicle” and “bundle” may be used interchangeably). In some embodiments, the clinical criteria used include the MEANS (Modular ECG Analysis System) criteria developed by the Department of Medical Informatics at the Erasmus University of Rotterdam in the Netherlands (Bemmel J H et al., “Methodology of the modular ECG analysis system MEANS”, Methods Inf Med. 1990 September;29(4):346-53), and/or other published criteria, e.g., as described by Surawicz et al. in “AHA/ACCF/HRRS Recommendations for the Standardization and Interpretation of the Electrocardiogram” (J. Amer. College of Cardio., 53:11, 2009 976-981).

It should be mentioned that the MEANS criteria are potentially a particularly good starting point for conversion to a graded indication of heart block status, as they have been formalized to explicitly link criteria in terms of Boolean operations and value comparisons. For example, a fragment of a MEANS criteria statement may read as follows:

```
1. skip tests if:
  Q wave in any of I, V5, V6
  or QRS duration <= 130 ms
2. say: “LBBB”
    if:
      QRS area in V1 < −100 mVms
      and S amplitude in V6 <= 1000 μV
    or −100 <= QRS area in V1 < −40 mVms
      and negative QRS amplitude > 3 times positive QRS amplitude in
V1
      and QRS area > 0 in V6
      and intrinsicoid deflection >= 50 ms in V5 or V6
```

The clinical criteria, e.g., the MEANS criteria, are defined generically, i.e., not personalized for a particular patient, and in particular not personalized based on measures of particular patient's pre-existing sub-threshold level of heart conduction block, if any. There may nevertheless be threshold values which vary generically as a function of a particular secondary factor, e.g., heart rate.

Criteria may be composed of a plurality of tests, including background conditions, qualitative tests, and quantitative tests related to durations and/or intervals above or below a specified time value. Values associated with tick 706 in each of indicators 701, 711, 721, 731 are selected based on a specified time value. Specified time values may themselves be variable dependent on associated factors. Standard block-assessment criteria may sometimes propose omitting a later part of a test due to an earlier part being clearly within a non-blocked range; however, in some embodiments of the present disclosure, these “shunting” instructions are ignored in order to obtain an assessment of sub-threshold block, and/or a baseline value. Other criteria affect validity of the block-assessment measurement (e.g., lack of fibrillation), and this sort of criterion is optionally retained.

As an example of criteria which is used, in some embodiments: first degree AV block (e.g., as would be indicated by indicator bar 731) is assessed to occur when a heart rate-dependent interval time between P wave and R wave onset in an ECG is exceeded. The heart rate-dependent interval may be based on a single value, e.g., 200 msec; or calculated, for example, using the following table:

| HEART RATE (BPM) | ≤70 | 71-90 | 91-110 | 111-130 | >130 |
|---|---|---|---|---|---|
| THRESHOLD INTERVAL | 220 | 210 | 200 | 190 | 180 |
| (MSEC) | | | | | |

Optionally, interpolation is used to select the heart block criterion value (e.g., as would be used to set the value of tick 706 of indicator bar 731); e.g., linear interpolation from 220 msec to 180 msec for heart rates between 70 BPM and 130 BPM.

A background condition based upon which the heart block criterion value is valid may be that there is no ongoing heart arrhythmia. In some embodiments, this background condition is considered met if there are at least, e.g., 10 seconds of normal sinus rhythm preceding a measurement of a threshold interval value. Optionally, alarms associated with indicator bar 731 are disabled in during ongoing heart arrhythmia; optionally, display of indicator bar 731 itself is disabled or diminished, e.g., by fading the indicator 731 to indicate that its value is presently invalid for the triggering of an alarm.

In another example, complete right bundle branch block (RBBB) (associated, for example, with tick 706 of indicator bar 701) is optionally associated with a QRS duration >120 msec; or if incomplete, 110-120 msec. Other criteria may be associated with particular leads of a 12-lead ECG; e.g., deflection features known as rsr′, rsR′, or rSR′ in leads V1 or V2 (sometimes referred to as a “bunny ear” pattern after its double-peaked shape); an S wave of greater duration than the R wave, or longer than 40 msec in lead I and/or V6 (in adults); and/or a normal RR peak time in leads V5 and V6 but >50 msec in lead V1. The extended S wave may also be referred to as “slurred” (e.g., mixing in with features after it that are normally distinct), as may be seen, e.g., in leads I, aVL, V5 and/or V6.

In another example, left branch bundle block (LBBB) may be evaluated based on an elongated QRS duration (e.g., >120 msec), associated with features such as broad notched or slurred RR waves in leads I, aVL, V5, and V6; absent q waves in leads I, V5 and V6; R peak time greater than 60 msec in leads V5 and V6 (but normal inn leads V1, V2 and V3); and/or opposite polarities of ST and T waves compared to the QRS.

In another example, left anterior fascicular block (LAFB) may be evaluated according to a QRS duration of less than 120 msec, and criteria such as an rS pattern in leads I and aVL, and/or a qR pattern in leads III and aVF.

### Baselines, Measurements, Calculations, and Display

The remaining blocks of FIG. 8 correspond, in some embodiments, to actions described in relation to FIG. 2.

At block 806, in some embodiments, baseline values are generated, for example as described in relation to block 206. Optionally, baseline values are taken from predetermined values; e.g., values measured from the patient at rest pre-operatively (the same day or within, e.g., one or two weeks beforehand), or values appropriate to the case of a current patient, selected from values established using measurements in a population of patients. The length of ECG recordings used to establish baseline values may comprise about 10 s of recorded data.

At block 808, in some embodiments, criteria inputs are measured, and graded results calculated. this may be performed, for example, as generally described in relation to blocks 212, 214 of FIG. 2, with the calculation being performed more particularly as is appropriate for one more groups of graded criteria, e.g., as described in relation to blocks 800-804.

At block 810, in some embodiments, the results of the actions of block 808 are displayed, for example, displayed as indicator bars or another display type, e.g., as described in relation to FIG. 7A-7B.

### Synthetic Lead Generation

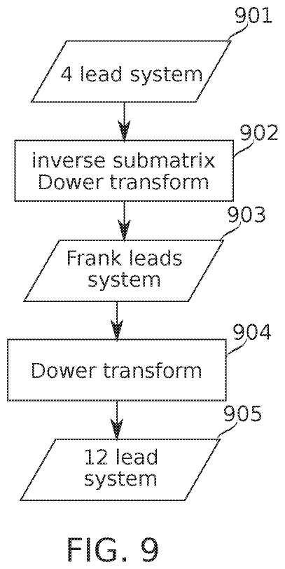

Reference is now made to FIG. 9, which is flow chart schematically illustrating a method of generating a synthetic 12-lead ECG from a reduced set of measurement electrodes, according to some embodiments of the present disclosure.

Criteria groups for heart block diagnosis often make reference to the particular leads of a 12-lead ECG (also known as the Einthoven-Wilson-Goldberger system or EWG system).

While the 12-lead ECG is a well-known standard among heart electrophysiology specialists, its use may be considered burdensome in the domain of structural heart disease specialists, due to the extra time, equipment, training, familiarity, area on a patient's body and/or complexity which a 12-lead ECG setup may impose on the already demanding procedures which they are called on to perform.

Accordingly, it is a potential advantage, in some embodiments, to provide a synthetic estimate of 12-lead ECG results using a reduced number of electrodes.

Even within a normal 12-lead ECG, it is normal for some leads to be “synthetic”, e.g., comprising results obtained by adding, subtracting, averaging, or otherwise combining recordings between a plurality of different sets of electrodes. An extensive literature exists which has also explored the interconversion of the standard 12-lead ECG system with the Frank lead system, used in vector cardiography. In a summary overview, there are three main methods for lead derivation:

1. Methods based on the Frank torso model, such as the well-known Dower and inverse Downer transforms (Dower, G. M. (1980). On Deriving the Electrocardiogram from Vectorcardiographic Leads. Clinical cardiology, 3(2), 87-95; Edenbrandt, L. &. (1988). Vectorcardiogram synthesized from a 12-lead ECG: superiority of the inverse Dower matrix. Journal of electrocardiology, 21(4), 361-367).

2. Statistical methods using linear regression to build desired leads from available ones.

3. Personalized regression, methods which perform regression for each patient.

The Dower/inverse Dower methods were developed to interconvert between the 10 electrodes of the 12-lead ECG, and the X-Y-Z axis leads of the Frank system.

In principle, the same methods can be applied to a four electrode ECG system comprising the arm/leg electrodes used to calculate leads I, II and optionally III, and a fourth electrode, for example V6 which is closer to the heart, but also somewhat out of the plane of leads I, II, and III so as to impart some information about the distribution of electrical fields in a third (depth) dimension. This provides a reasonable approximation to the three axis leads of the Frank system.

The procedure, for generating a 12-lead ECG system, in some embodiments, is outlined in FIG. 9.

At block 902, in some embodiments, the inverse Dower transform is applied to leads of the 4-electrode system 901 by calculating the inverse of the submatrix of the Dower transform composed of rows corresponding to the available channels. In effect, the 12-lead transform matrix is used, but only “filled in” for the channels available.

This yields block 903, the estimated Frank leads.

From there, the normal Dower lead transform is applied, to generate a 12-lead system from the estimated Frank leads.

It should be understood that this does not generate “something from nothing”; information in the 12-lead system generated according to the method of FIG. 9 cannot supply information missing in the original 4-lead data. The utility, for some embodiments of the present invention, is that it creates leads which are in a format, albeit potentially a somewhat degraded one, corresponding to format which the diagnostic criteria groups relying on the 12-lead system describe.

### General

It is expected that during the life of a patent maturing from this application many relevant structural heart disease interventions will be developed; the scope of the term structural heart disease intervention is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

In addition, any priority document(s) of this application is/are hereby incorporated herein by reference in its/their entirety.

1-24. (canceled)

25. A device for intraoperative monitoring of new onset conduction disturbances, the device comprising:
an indicator device configured to present an alarm responsive to alarm instruction;
a processor coupled to the indicator device; and
a digital memory accessible to the processor and storing criteria for determining existence of a heart conduction block based on ECG data;
wherein the memory also stores instructions instructing the processor to:
access a portion of an ongoing ECG signal recording recorded from a patient during the performance of a transcatheter heart procedure;
calculate a test value of a metric indicative of a level of partial heart conduction block from the portion of the ongoing ECG recording using baseline ECG data recorded from the patient and the stored criteria; and
send an alarm instruction to the indicator device based on the calculated test value.

26. The device of claim 25, together with ECG circuitry configured to record the ECG signal recording.

27. The device of claim 25, wherein the digital memory stores the baseline ECG data.

28. The device of claim 25, wherein the instructions instruct the processor to:
transform threshold parameters used in the criteria into range parameters; and
calculate the test value based on the range parameters.

29. The device of claim 25, wherein the instructions instruct the processor to transform a threshold parameter used in the criteria to a range parameter by establishing a range between a baseline value and a threshold value, wherein the threshold value is used in the criteria and the baseline value is derived from the baseline ECG data using the rules.

30. The device of claim 25, wherein the stored criteria are for determining existence of a heart conduction block in a heart of any patient based on ECG data.

31. The device of claim 28, wherein the instructions instruct the processor to calculate the test value by combining the range parameters into a single range parameter having:
a minimum value when each of the range parameters has a value equal to its baseline value, and
a maximum value when the predetermined criteria indicate the existence of the heart conduction block.

32. The device of claim 28, wherein the instructions instruct the processor to calculate the test value by combining the range parameters into a single range parameter having:
a maximum value when each of the range parameters has a value equal to its baseline value, and
a minimum value when the predetermined criteria indicate the existence of the heart conduction block.

33. The device of claim 28, wherein the alarm instruction is indicative of heart conduction block at a specific structure of the heart conduction system.

34. The device of claim 33, wherein the indicator device displays a visual representation of a heart conduction system with a mark presented at the specific structure of the heart conduction system and with a visual characteristic indicative of the alarm instruction.

35. The device of any one of claim 25, wherein the stored predetermined refer rules to leads of a standard 12-lead ECG.

36. The device of claim 35, wherein the ongoing ECG signal recording is obtained using a set of electrodes having less electrodes than defined for performing a standard 12-lead ECG, and the device comprises an interface connecting to electrodes with fewer electrode attachment ports than ten.

37. The device of claim 36, including the set of electrodes, wherein the set of electrodes consists of four electrodes.

38. The device of any one of claim 36, wherein the digital memory stores the baseline ECG data, collected using standard 12-lead ECG.

39. The device of claim 38, wherein the four electrodes correspond to the arm and leg electrodes of the standard 12-lead ECG, and one of the chest electrodes.

40. The device of claim 39, wherein the chest electrode is the V6 electrode.

41. The device of claim 36, wherein the instructions instruct the processor to transform the ongoing ECG data recorded from the patient into a standard ECG data referring to leads corresponding to a standard 12-lead ECG.

42. The device of claim 41, wherein the processor transforms the ongoing ECG data by performing a modified inverse Dower transform to produce a set of leads corresponding to Frank vector cardiography leads, and a Dower transform on the produced set of leads to yield the 12-lead ECG.

43. The device of claim 25, wherein the indicator device presentation of the alarm in response to the alarm instruction comprises display of a vector electrogram, and a change thereto.

44. The device of claim 25, wherein the indicator device presentation of the alarm in response to the alarm instruction is indicative of a potential to halt or reverse the partial heart conduction block.

45. The device of claim 25, wherein the memory stores instructions instructing the processor to:
calculate a test value of a metric indicative of a level of heart conduction disturbance from the portion of the ongoing ECG recording using baseline ECG data recorded from the patient and the stored criteria; and
send an alarm instruction to the indicator device based on the calculated test value indicative of heart conduction disturbance.

46. The device of claim 25, wherein an interval between the portion of the ongoing ECG recording and the baseline ECG data is a single heartbeat.

47. The device of claim 25, wherein the processor calculates test values for a plurality of metrics indicative of the level of partial heart conduction block, and sends alarm instructions based thereon;
wherein the plurality of metrics includes a metric derived from a first combination epoch, and from a second combination epoch, longer than the first combination epoch.
